(19) United States
(12) Patent Application Publication
(10) Pub. No.: US 2024/0257915 A1
(43) Pub. Date: Aug. 1, 2024

(54) ONLINE BASE CALL COMPRESSION

(71) Applicant: Roche Sequencing Solutions, Inc., Pleasanton, CA (US)

(72) Inventors: John MANNION, Mountain View, CA (US); James HAN, San Carlos, CA (US); Miroslav KUKRICAR, Dublin, CA (US); Denis TOLKUNOV, Dublin, CA (US)

(21) Appl. No.: 18/625,006

(22) Filed: Apr. 2, 2024

Related U.S. Application Data
(63) Continuation of application No. PCT/US22/45624, filed on Oct. 4, 2022.
(60) Provisional application No. 63/251,979, filed on Oct. 4, 2021.

(51) Int. Cl. G16B 30/10

(52) U.S. Cl. CPC G16B 30/10

(57) ABSTRACT

For high sequencing throughput, circuitry can compress read data generated in real-time by a sequencing device. Various compression techniques can be used. A stream of raw data can be processed to generate raw read data stream. The raw read data stream may include sub-streams of data comprising a header data sub-stream, a basecall sub-stream, and a quality score sub-stream. The sub-streams can be extracted and compressed using separate threads, and the compressed data can be recombined. Sequence reads corresponding to different copies of the same nucleic acid molecule may be clustered and used to generate a consensus read. The number of sequence reads that are used to generate the consensus read can be limited to a threshold when a consensus read is substantially accurate. After the limit is reached, data from any new raw read data corresponding to the same nucleic acid molecule may be discarded.

### CROSS-REFERENCE TO RELATED PATENT APPLICATIONS

The present application is a U.S. Bypass Continuation Application of International Application PCT/US2022/045624 filed Oct. 4, 2022, which claims benefit of priority to U.S. Provisional Patent Application No. 63/251,979, filed Oct. 4, 2021, which are incorporated herein by reference for all purposes.

### BACKGROUND

A sequencing device such as the nanopore devices can be used for rapid sequencing of nucleic acids in biological samples. The sequencing device can generate raw data corresponding to signals associated with detecting nucleotides (directly or indirectly) in a nucleic acid molecule from the biological sample. The raw data produced by the sensors in the device can then be transformed into raw read data (e.g., by another part of a sequencing system) that corresponds to determining the type and the order of the detected nucleotides in a sequenced molecule. Determining the type of the nucleotide and its order in the sequence of nucleotides is also known as base calling. The raw read data can comprise other information such as data associated with the quality of the signal collected.

Improving the capability of the sequencing device to detect signals at a faster rate translates to generating large amounts of raw data. Consequently, a large amount of raw read data can also be generated, which can cause problems such as bottlenecks that can constrain the rate of signals, thereby limiting the throughput of the sequencing.

### SUMMARY

The present disclosure relates generally to nucleic acid sequencing, and more specifically, to embodiments that can enable high sequencing throughput. For example, some embodiments (e.g., inference circuitry) can compress read data generated using raw data received from a sequencing device (e.g., nanopore-based sequencing devices). Various compression techniques can be used such that the amount of output data is decreased, so that an output bottleneck does not cause errors or to artificially limit the speed at which a sequencing device can operate.

According to one embodiment, raw data can be received from a sensor chip including a plurality of cells. The raw data can include a plurality of measurements for each position of a nucleic acid molecule. The raw data can include measurements of at least 100.000 nucleic acid molecules. A read data stream can be generated that includes header information, basecall data, and quality scores for the nucleic acid molecules. A first sub-stream of header information can be extracted from the read data stream. The header information can identify each of the nucleic acid molecules. Compressed header information can be generated by compressing the first sub-stream of header information, using a first thread. A second sub-stream of basecall data can be extracted from the read data stream. The basecall data sub-stream can provide a basecall at each position of each of the nucleic acid molecules. Compressed basecall data can be generated by compressing the second sub-stream of basecall data, using a second thread. A third sub-stream of quality score data can be extracted from the read data stream. The quality score data can provide a quality score for each basecall at each position of each of the nucleic acid molecules. Compressed quality score data can be generated by compressing the third sub-stream of quality score data, using a third thread. In various implementations, the sub-streams of data can be output separate or combined and then output. For example, two or more of the compressed header information, the compressed basecall data, and the compressed quality score data can be combined to generate a stream of compressed data. The stream of compressed data can then be output.

In some embodiments for compressing raw read data, a sequence read from the sub-stream of basecall data corresponding to a template nucleic acid molecule can be aligned to a reference sequence (e.g., a reference genome). The reference sequence may comprise a naturally occurring (e.g., human genome) or synthetic nucleic acid sequence (e.g., genetically engineered DNA or RNA). The synthetic sequence may comprise naturally occurring or synthetic amino acids (e.g., amino acids containing synthetic nucleoside and/or nucleotide analogues). A location of the sequence read can be determined relative to the reference sequence. Similarities and differences between the sequence read from the basecall data and the reference sequence can be identified for each nucleotide. The sequence read can be encoded using codes generated based on the identified similarities and differences. The encoded sequence read can then be compressed using patterns within the code(s) of the encoded sequence (e.g., a repeated code or sequence of codes) and the genomic location 30) information. At least a portion of the sequence (e.g., base pair type) information in the sequence reads from the basecall data sub-stream can be replaced with the genomic location information (i.e., the genomic location corresponding to the reference) when the read information matches the reference, and codes for differences can be used for nucleotides that do not match. Accordingly, the location information can substitute the sequence read information for at least a portion of the sequence that matches the reference sequence in a consecutive manner.

The sub-stream of quality score data corresponding to the sequence read from the basecall data can also be encoded and compressed accordingly. The encoding of the quality score data may not require a reference genome. For example, the quality score data may be compressed by transforming discrete (or quantitative) quality scores to concrete (or qualitative) quality scores (e.g., categorical data). Additional details regarding quality score compression is provided below.

The genomic locations of the reads and the codes can be generated in real-time, along with the compression of the codes. The inference circuitry used to determine the genomic locations and the codes can include a local memory that stores data temporarily for processing. The local memory can be a memory associated with the inference circuitry, which may be on the same integrated circuit or connected via a high throughput bus. The inference circuitry (e.g., to perform the steps of aligning and storing) can include, for example, a graphics processing unit (GPU), field programmable gate arrays (FPGAs), a central computing unit (CPU), or a combination thereof. Other processing units may be used to perform the methods mentioned herein.

In some embodiments, the first sub-stream of header information, the second sub-stream of basecall data, and the third sub-stream of quality score data can be compressed simultaneously. Different portions of the computational resources (e.g., CPU, GPU, FPGA processing units, memory, etc.), can be assigned to each of the sub-streams. A size of each the portions of the computational resources allocated to process each of the sub-streams can be managed by a load-balancing system. The load-balancing system can be optimized so that each of the sub-streams are compressed during roughly the same period of time such that the final output is synchronized, with the compressed header data, read data, and quality score data for a given nucleic acid ready for output at the same time.

In some embodiments for clustering sequence reads, a consensus sequence read can be generated for a template nucleic acid molecule based on two or more sequence reads corresponding to copies of the template nucleic acid molecule. The consensus sequence reads can be generated before or after the sequence reads are clustered. The consensus sequence reads can be generated for each cluster as new sequence reads are assigned to the cluster, or the consensus sequence reads can be generated after the number of sequence reads in the cluster reaches the threshold before or after outputting the sequence reads of the cluster. The sequence reads corresponding to the same template may be clustered together, as described above and elsewhere herein, or can be identified based on barcodes and/or location information (e.g., as a result of aligning) of the two or more sequence reads, thereby identifying the sequence reads as corresponding to the same nucleic acid molecule or a molecular family. The two or more sequence reads can be compiled into one consensus read, which can be done on the inference circuitry or later circuitry in the pipeline. When done on the inference circuitry, the consensus sequence read can evolve as more raw data from the same nucleic acid molecule or molecular family is generated. The consensus sequence read can be compressed based on location and code (e.g., encoding nucleotides based on an alignment information) generated for each nucleic acid (e.g., DNA base, or RNA base) compared to a reference genome, as described above and elsewhere herein.

A cutoff amount (threshold) can be determined for the number of sequence reads that are used to generate a consensus sequence read for a nucleic acid molecule or a molecular family. In this manner, fewer sequence reads may need to be output from the inference circuitry when the consensus read is determined by later circuitry, since sequence reads above the cutoff amount can be discarded. Such discarding can be beneficial when certain template nucleic acids are amplified too much (e.g., during PCR prior to sequencing). Or, if the consensus is generated by the inference circuitry, computational resources and memory can be saved by not using all of the sequence reads for a nucleic acid molecule to build the consensus, but instead only using a sufficient number. A consensus sequence read for a nucleic acid molecule or molecular family can be substantially generated in such a manner. The cutoff value may correspond to the threshold associated with clustering, as described above or elsewhere herein.

According to one embodiment, raw data can be received from a sensor chip including a plurality of cells. The raw data can include a plurality of measurements for each position of a nucleic acid molecule. The raw data can include measurements of at least 100,000 nucleic acid molecules. A portion of the at least 100,000 nucleic acid molecules can include clusters of nucleic acid molecules. The clusters of nucleic acid molecules can be generated by making copies of the template nucleic acid molecule. The copies can be made using polymerase chain reaction (PCR). The nucleic acid molecules of a cluster can correspond to a same template nucleic acid molecule. Sequence data can be generated by an inference circuitry from the raw data of a nucleic acid molecules by determining a nucleotide for each position in the sequence of the nucleic acid molecule. Sequence reads of the at least 100,000 nucleic acid molecules can then be clustered. A counter can keep a count of a size of each cluster (e.g., the number of sequence reads that are assigned into a cluster). The size of a cluster may be capped at a particular threshold (cutoff amount). Therefore, as each sequence read is assigned to a particular cluster corresponding to the sequence read a counter increment for that cluster increases (i.e., by one). The counter for the cluster can then be compared to a predetermined threshold. If the counter is greater than the threshold, the sequence read assigned to the cluster can be discarded (i.e., removed from the memory). When the counter is smaller than the threshold, the sequence read can be added to sequence reads corresponding to the cluster. Sequence reads corresponding to a cluster with a counter equal or greater than the threshold can be output. The output can be transmitted to a memory device (e.g., a disk, a cloud-based storage, etc.). For each cluster a consensus read may be generated based on the sequence reads assigned to each cluster. The consensus read may then be compressed and output from the sequencing system (e.g., to a storage device).

In some embodiments for clustering sequence reads, a sequence read can include one or more barcode sequences corresponding to nucleotides attached to the nucleic acid molecule. A particular cluster can be assigned to one or more particular barcode sequences. Identifying a particular cluster corresponding to the sequence read can include comparing one or more barcode sequences of the sequence read to the one or more particular barcode sequences, that one or more clusters are assigned to, to determine a match. A cluster can be created for a new sequence read when the one or more barcode sequences of the new sequence read do not match to any of the barcode sequences that existing clusters are assigned to. Identifying the particular cluster corresponding to the sequence read can also include comparing the content of the sequence read with a sequence content that each cluster is assigned to (e.g., similar to comparing a barcode sequence). For example, this may be performed by aligning the sequence read to a reference genome to determine a genomic location. The genomic location can then be compared to one or more genomic locations that one or more clusters are assigned to. The genomic location can include a start genomic location and an end genomic location. The genomic location of a particular cluster can be determined using another sequence read of the particular cluster (e.g., by pairwise or multiple alignment between the content of a sequence read and the sequence reads in a particular cluster).

These and other embodiments of the invention are described in detail below. For example, other embodiments are directed to systems, devices, and computer readable media associated with methods described herein.

A better understanding of the nature and advantages of embodiments of the present invention may be gained with reference to the following detailed description and the accompanying drawings.

### BRIEF DESCRIPTION OF THE DRAWINGS

FIG. 1 illustrates an embodiment of a cell in a nanopore based sequencing chip.

FIG. 2 illustrates an embodiment of a cell in a nanopore based sequencing chip.

FIG. 3 illustrates an embodiment of a cell performing nucleotide sequencing with the Nano-SBS technique.

FIG. 4 illustrates an embodiment of a cell about to perform nucleotide sequencing with pre-loaded tags.

FIG. 5 illustrates an embodiment of a sequencing process with pre-loaded tags.

FIG. 6A illustrates an embodiment of a circuitry in a cell of a nanopore based sequencing chip, wherein the circuitry can be configured to detect whether a lipid bilayer is formed in the cell without causing an already formed lipid bilayer to break down.

FIG. 6B illustrates the same circuitry in a cell of a nanopore based sequencing chip as that shown in FIG. 6A. Comparing to FIG. 6A, instead of showing a lipid membrane/bilayer between the working electrode and the counter electrode, an electrical model representing the electrical properties of the working electrode and the lipid membrane/bilayer is shown.

FIG. 7 shows example data points captured from a nanopore cell during bright periods and dark periods of AC cycles.

FIG. 8 illustrates an embodiment of a sequencing instrument hardware configuration according to certain embodiments.

FIG. 9 shows a flow chart illustrating an example method of compressing raw read data according to certain embodiments.

FIG. 10 shows a flow chart illustrating an example method of compressing read data stream using multiple threads according to certain embodiments.

FIG. 11A illustrates an embodiment of a raw read data compression system according to certain embodiments. FIG. 11B shows an example for when the threads are software threads that can be scheduled on one or more processing units according to embodiments of the present disclosure.

FIG. 12 shows a flow chart illustrating an example method of compressing a sub-stream of basecall data according to certain embodiments.

FIG. 13-18 show experimental results of compressing sequencing data according to certain embodiments.

FIG. 19 illustrates an example of an amplification process according to certain embodiments.

FIG. 20 illustrates an embodiment of a sequence read data clustering system according to certain embodiments.

FIG. 21 shows a flow chart illustrating an example method of clustering read data to reduce an amount of sequencing data according to certain embodiments.

FIG. 22 shows the raw data for multiple passes of a molecule (e.g., an xpandomer molecule) being read using a nanopore according to certain embodiments.

FIG. 23 illustrates sequencing to generate an intramolecular consensus according to embodiments of the present invention.

FIG. 24 shows a block diagram of an example computer system usable with system and methods according to certain embodiments.

### DEFINITIONS

“Nucleic acid” may refer to deoxyribonucleotides or ribonucleotides and polymers thereof in either single- or double-stranded form. The term may encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs may include, without limitation, phosphorothioates, phosphoramidites, methyl phosphonates, chiral-methyl phosphonates, 2-O-methyl ribonucleotides, peptide-nucleic acids (PNAs). The nucleic acid may also be represented by surrogate molecules, which are inserted into the original nucleic acid, with each surrogate molecule corresponding to a particular nucleotide.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991): Ohtsuka et al., J. Biol. Chem. 260:2605-2608 (1985): Rossolini et al., Mol. Cell. Probes 8:91-98 (1994)). The term nucleic acid is used interchangeably with gene, cDNA, mRNA, oligonucleotide, and polynucleotide.

The term “nucleotide,” in addition to referring to the naturally occurring ribonucleotide or deoxyribonucleotide monomers, may be understood to refer to related structural variants thereof, including derivatives and analogs (e.g., X-NTPs used in SBX-sequencing), that are functionally equivalent with respect to the particular context in which the nucleotide is being used (e.g., hybridization to a complementary base), unless the context clearly indicates otherwise.

The term “tag” may refer to a detectable moiety that can be atoms or molecules, or a collection of atoms or molecules. A tag can provide an optical, electrochemical, magnetic, or electrostatic (e.g., inductive, capacitive) signature, which signature may be detected with the aid of a nanopore. Typically, when a nucleotide is attached to the tag it is called a “Tagged Nucleotide.” The tag can be attached to the nucleotide via the phosphate moiety.

The term “raw data” or “raw signal data” refers to data produced by sensors in a sequencing device. Raw data includes signal values associated with sequencing a nucleic acid molecule.

“Nanopore” refers to a pore, channel or passage formed or otherwise provided in a membrane. A membrane can be an organic membrane, such as a lipid bilayer, or a synthetic membrane, such as a membrane formed of a polymeric material. The nanopore can be disposed adjacent or in proximity to a sensing circuit or an electrode coupled to a sensing circuit, such as, for example, a complementary metal oxide semiconductor (CMOS) or field effect transistor (FET) circuit. In some examples, a nanopore has a characteristic width or diameter on the order of 0.1 nanometers (nm) to about 1000 nm. Some nanopores are proteins.

The term “bright period” may generally refer to the time period when a tag of a tagged nucleotide is forced into a nanopore by an electric field applied through an AC signal. The term “dark period” may generally refer to the time period when a tag of a tagged nucleotide is pushed out of the nanopore by the electric field applied through the AC signal. An AC cycle may include the bright period and the dark period. In different embodiments, the polarity of the voltage signal applied to a nanopore cell to put the nanopore cell into the bright period (or the dark period) may be different. The bright periods and the dark periods can correspond to different portions of an alternating signal relative to a reference voltage.

The term “signal value” may refer to a value of the sequencing signal output from a sequencing cell. According to certain embodiments, the sequencing signal may be an electrical signal that is measured and/or output from a point in a circuit of one or more sequencing cells e.g., the signal value may be (or represent) a voltage or a current. The signal value may represent the results of a direct measurement of voltage and/or current and/or may represent an indirect measurement, e.g., the signal value may be a measured duration of time for which it takes a voltage or current to reach a specified value. A signal value may represent any measurable quantity that correlates with the features of the sequencing device. For example, in a nanopore sequencing device the resistivity of a nanopore and from which the resistivity and/or conductance of the nanopore (threaded and/or unthreaded) may be derived can affect the signal value. As another example, the signal value may correspond to a light intensity, e.g., from a fluorophore attached to a nucleotide being catalyzed to a nucleic acid with a polymerase.

The term “raw read data” or “read data” refers to data generated from the raw data or the raw signal data. The raw read data includes read data stream(s). A read data stream includes sub-streams of data corresponding to a respective nucleic acid molecule including an identifier or header sub-stream, a nucleic acid basecall sub-stream, and a quality score sub-stream.

The term “basecall data” refers to data generated from the raw data that identifies a nucleotide (e.g., a nitrogen-containing base of a nucleotide) at a given location in a nucleic acid sequence. Each entry in a basecall data represents a nucleotide and can include one code for the corresponding nucleotide. The basecall data can include primary nucleotides such as adenine (A), thymine (T), guanine (G), cytosine (C), and uracil (U) or a synthetic nucleotide. The basecall data may also include other possible base calls such as an undetermined nucleotide.

The term “quality score data” refers to data generated from the raw data that provides a measure for confidence in accuracy of a basecall correctly made for a nucleic acid (e.g., between the four bases.) The quality score can be reflective of the stochastic behavior that is inherent to single molecule observations. The quality of basecalls may not degrade with time or with read length, but there can be different quality scores for different basecalls randomly at different points in time on a given nucleic acid. Alternatively, the quality scores of bases in a read may show a dependence on read length or position of base within a read. A higher quality score for a basecall can indicate greater confidence in the basecall being correct. For example, a signal value that is near a peak of a probability distribution function (PDF) can result in a basecall having a higher quality score than a signal value that is far from a peak of a PDF.

The term “header data,” “read ID data” refers to information that identifies a read within a larger collection of reads. For example, the raw read data stream generated for a portion of the raw data has the same header data across the raw read data stream for that portion. The raw data can include a plurality of portions of raw data generated simultaneously or at different times for the same nucleic acid molecule (e.g., template nucleic acid molecule) or for different nucleic acid molecules (e.g., different template nucleic acid molecules).

The term “consensus sequence read.” “consensus sequence.” “consensus read.” or “consensus” refers to a nucleic acid sequence read generated from aligning a plurality of sequence reads that correspond to the same template nucleic acid molecule or molecular family. The consensus sequence read may be generated by aligning the plurality of sequence reads to one another. Or, by aligning each of the plurality of sequence reads to a reference genome.

The term “real-time” or “live” refers to processing raw data from a nucleic acid molecule at a rate equal or great than the raw data is generated. Real-time processing of the raw data eliminates the need to store raw data or read data in a long term memory (e.g., disc, hard drive, cloud storage, or any external memory device).

### DETAILED DESCRIPTION

Techniques disclosed herein relate to analyzing sequencing data of one or more nucleic acid molecules generated from a sequencing device, and more specifically, to efficiently processing (e.g., compressing, filtering, or discarding) sequence read data generated by the sequencing device (e.g., nanopore-based sequencing device). The sequencing device can generate raw data at a very high rate. The raw data may be processed (e.g., by another part of a sequencing system) to provide an output that includes a sequence information (e.g., RNA or DNA sequence) of the nucleic acid molecule, referred to as raw read data. Any bottlenecks in transmitting and/or storing of this output can limit the throughput of the sequencing. Therefore, to transmit and store the output at a rate equivalent to the raw data generation of the sequencing device, the output needs to be processed and compressed in real-time. The compressed data can then transmitted out of the sequencing device, for example, to be stored in a storage device.

In some cases, a series of sequencing processes are performed on the same sequencing device, e.g., different sequencing runs with new DNA molecules in each cell. The time in between two consecutive sequencing processes or turnaround time may be insufficient to offload the raw data generated at each sequencing process from the channels downstream the sequencing device. Therefore, analyzing and compressing data generated in each sequencing process may be performed in real-time as the data is generated. This may allow storing the compressed data to be completed before or during the turnaround time.

A stream of raw data can be processed (e.g., by an inference chip) to generate raw read data stream. The raw read data stream may include sub-streams of data comprising a header data sub-stream, a basecall sub-stream, and a quality score sub-stream. The header data may comprise information that can identify a raw read data stream and its sub-streams corresponding to a nucleic acid molecule and other information corresponding to the sequencing device and the sequencing process (e.g., sequencing device information, time of the sequencing, etc.). The basecall data sub-stream can comprise nucleotide information (i.e., base call codes for a nucleotide) for each corresponding position in a sequence read. The quality score data sub-stream may comprise a confidence value for each basecall corresponding to each nucleotide in the sequence read form the basecall data sub-stream. The sub-streams can be extracted and compressed using separate threads. In some implementations, the compressed data can be recombined.

In some embodiments, a sequence read from a basecall data sub-stream of a raw read data stream is compressed by means of aligning the sequence read to a reference genome. The sequence read can be encoded by replacing the nucleotides in a sequence read with the alignment information. The encoding can distinguish if a nucleotide from the sequence read matches the reference genome sequence or if there is a mismatch. The mismatch can comprise insertions, deletions, skips, or soft-clips The encoding and the location of each nucleotide relative to the reference genome can be used to compress the sequence read. For example, a series of matched nucleotides can be compressed to a range of locations with a beginning and an end location relative to the reference genome.

In some embodiments, template nucleic acid molecules may be amplified during library preparation prior to sequencing. Thus, multiple nucleic acid molecules (e.g., copies and original) of the template can be sequenced. Then, raw data corresponding to these nucleic acid molecules or portions thereof may be generated by the sequencing device (e.g., at different time points). Sequence reads (e.g., from raw read data) of two or more raw data corresponding to different copies of the same nucleic acid molecule may be clustered and used to generate a consensus read for the nucleic acid molecule. The number of sequence reads that are used to generate the consensus read can be limited to a cutoff number (threshold) or until a consensus read is considered complete or substantially accurate. After the limit/cutoff is reached, data from any new raw read data that corresponds to the same nucleic acid molecule or portions thereof may be discarded and excluded from further analysis. The corresponding new raw read data may be removed from the instrument to reduce the amount of data in the memory and the amount of data that needs to be output from the memory.

### I. Nanopore System

A nanopore cells in nanopore sensor chip may be implemented in many different ways. For example, in some embodiments, tags of different sizes and/or chemical structures may be attached to different nucleotides in a nucleic acid molecule to be sequenced. In some embodiments, a complementary strand to a template of the nucleic acid molecule to be sequenced may be synthesized by hybridizing differently polymer-tagged nucleotides with the template. In some implementations, the nucleic acid molecule and the attached tags may both move through the nanopore, and an ion current passing through the nanopore may indicate the nucleotide that is in the nanopore because of the particular size and/or structure of the tag attached to the nucleotide. In some implementations, only the tags may be moved into the nanopore. There may also be many different ways to detect the different tags in the nanopores.

### A. Nanopore Sequencing Cell

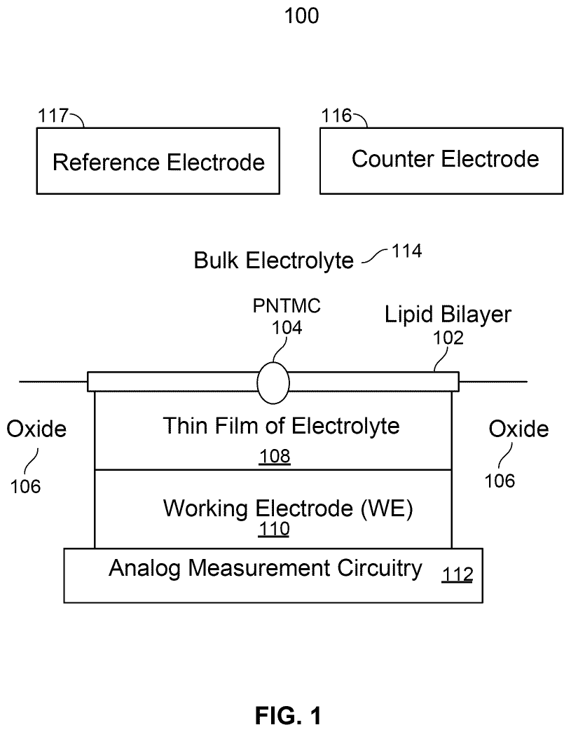

FIG. 1 is a simplified structure illustrating an embodiment of a nanopore cell 100 in a nanopore based sequencing chip, according to certain embodiments. Nanopore cell 100 may include a well formed by dielectrical material, such as oxide 106. A membrane 102 may be formed over the surface of the well to cover the well. In some embodiments, membrane 102 may be a lipid bilayer. A bulk electrolyte 114 that may contain, for example, soluble protein nanopore transmembrane molecular complexes (PNTMC) and the analyte of interest, is placed onto the surface of the cell. A single PNTMC 104 may be inserted into membrane 102 by electroporation. The individual membranes in an array are neither chemically nor electrically connected to each other. Thus, each cell in the array is an independent sequencing machine, producing data unique to the single polymer molecule associated with the PNTMC. PNTMC 104 operates on the analytes and modulates the ionic current through the otherwise impermeable bilayer.

Analog measurement circuitry 112 is connected to a working electrode 110 (e.g., composed of metal) covered by a thin film of electrolyte 108. The thin film of electrolyte 108 is isolated from the bulk electrolyte 114 by membrane 102 that is ion-impermeable. PNTMC 104 crosses membrane 102 and provides the only path for ionic current to flow from the bulk liquid to working electrode 110. The cell also includes a counter electrode (CE) 116, which is an electrochemical potential sensor. The cell also includes a reference electrode 117.

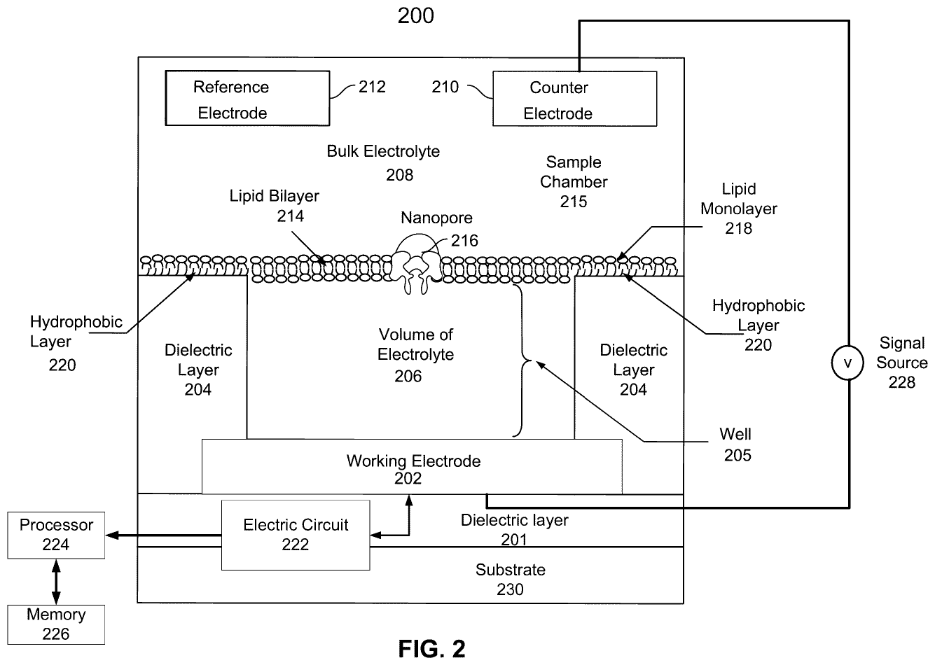

FIG. 2 illustrates an embodiment of an example nanopore cell 200 in a nanopore sensor chip that can be used to characterize a polynucleotide or a polypeptide, according to certain embodiments. Nanopore cell 200 may include a well 205 formed of dielectric layers 201 and 204: a membrane, such as a lipid bilayer 214 formed over well 205; and a sample chamber 215 on lipid bilayer 214 and separated from well 205 by lipid bilayer 214. Well 205 may contain a volume of electrolyte 206, and sample chamber 215 may hold bulk electrolyte 208 containing a nanopore, e.g., a soluble protein nanopore transmembrane molecular complexes (PNTMC), and the analyte of interest (e.g., a nucleic acid molecule to be sequenced).

Nanopore cell 200 may include a working electrode 202 at the bottom of well 205 and a counter electrode 210 disposed in sample chamber 215. A signal source 228 may apply a voltage signal between working electrode 202 and counter electrode 210. A single nanopore (e.g., a PNTMC) may be inserted into lipid bilayer 214 by an electroporation process caused by the voltage signal, thereby forming a nanopore 216 in lipid bilayer 214. The individual membranes (e.g., lipid bilayers 214 or other membrane structures) in the array may be neither chemically nor electrically connected to each other. Thus, each nanopore cell in the array may be an independent sequencing machine, producing data unique to the single polymer molecule associated with the nanopore that operates on the analyte of interest and modulates the ionic current through the otherwise impermeable lipid bilayer.

As shown in FIG. 2, nanopore cell 200 may be formed on a substrate 230, such as a silicon substrate. Dielectric layer 201 may be formed on substrate 230. Dielectric material used to form dielectric layer 201 may include, for example, glass, oxides, nitrides, and the like. An electric circuit 222 for controlling electrical stimulation and for processing the signal detected from nanopore cell 200 may be formed on substrate 230 and/or within dielectric layer 201. For example, a plurality of patterned metal layers (e.g., metal 1 to metal 6) may be formed in dielectric layer 201, and a plurality of active devices (e.g., transistors) may be fabricated on substrate 230. In some embodiments, signal source 228 is included as a part of electric circuit 222. Electric circuit 222 may include, for example, amplifiers, integrators, analog-to-digital converters, noise filters, feedback control logic, and/or various other components. Electric circuit 222 may be further coupled to a processor 224 that is coupled to a memory 226, where processor 224 can analyze the sequencing data to determine sequences of the polymer molecules that have been sequenced in the array.

Working electrode 202 may be formed on dielectric layer 201, and may form at least a part of the bottom of well 205. In some embodiments, working electrode 202 is a metal electrode. For non-faradaic conduction, working electrode 202 may be made of metals or other materials that are resistant to corrosion and oxidation, such as, for example, platinum, gold, titanium nitride, and graphite. For example, working electrode 202 may be a platinum electrode with electroplated platinum. In another example, working electrode 202 may be a titanium nitride (TiN) working electrode. Working electrode 202 may be porous, thereby increasing its surface area and a resulting capacitance associated with working electrode 202. Because the working electrode of a nanopore cell may be independent from the working electrode of another nanopore cell, the working electrode may be referred to as cell electrode in this disclosure.

Dielectric layer 204 may be formed above dielectric layer 201. Dielectric layer 204 forms the walls surrounding well 205. Dielectric material used to form dielectric layer 204 may include, for example, glass, oxide, silicon mononitride (SiN), polyimide, or other suitable hydrophobic insulating material. The top surface of dielectric layer 204 may be silanized. The silanization may form a hydrophobic layer 220 above the top surface of dielectric layer 204. In some embodiments, hydrophobic layer 220 has a thickness of about 1.5 nanometer (nm).

Well 205 formed by dielectric layer 204 includes volume of electrolyte 206 above working electrode 202. Volume of electrolyte 206 may be buffered and may include one or more of the following: lithium chloride (LiCl), sodium chloride (NaCl), potassium chloride (KCl), lithium glutamate, sodium glutamate, potassium glutamate, lithium acetate, sodium acetate, potassium acetate, calcium chloride (CaCl₂)), strontium chloride (SrCl₂), manganese chloride (MnCl₂), and magnesium chloride (MgCl₂). In some embodiments, volume of electrolyte 206 has a thickness of about three microns (μm).

As also shown in FIG. 2, a membrane may be formed on top of dielectric layer 204 and span across well 205. In some embodiments, the membrane may include a lipid monolayer 218 formed on top of hydrophobic layer 220. As the membrane reaches the opening of well 205, lipid monolayer 218 may transition to lipid bilayer 214 that spans across the opening of well 205. The lipid bilayer may comprise or consist of phospholipid, for example, selected from diphytanoyl-phosphatidylcholine (DPhPC), 1,2-diphytanoyl-sn-glycero-3-phosphocholine, 1,2-Di-O-Phytanyl-sn-Glycero-3-phosphocholine (DoPhPC), palmitoyl-oleoyl-phosphatidylcholine (POPC), dioleoyl-phosphatidyl-methy lester (DOPME), dipalmitoylphosphatidylcholine (DPPC), phosphatidylcholine, phosphatidylethanolamine, phosphatidylserine, phosphatidic acid, phosphatidylinositol, phosphatidylglycerol, sphingomyelin, 1,2-di-O-phytanyl-sn-glycerol; 1,2-dipalmitoyl-sn-glycero-3-phosphoethanolamine-N-[methoxy (polyethylene glycol)-350], 1,2-dioleoyl-sn-glycero-3-phosphoethanolamine-N-lactosyl; GMI Ganglioside, Lysophosphatidylcholine (LPC) or any combination thereof.

As shown, lipid bilayer 214 is embedded with a single nanopore 216, e.g., formed by a single PNTMC. As described above, nanopore 216 may be formed by inserting a single PNTMC into lipid bilayer 214 by electroporation. Nanopore 216 may be large enough for passing at least a portion of the analyte of interest and/or small ions (e.g., Na⁺, K⁺, Ca²⁺, CI⁻) between the two sides of lipid bilayer 214.

Sample chamber 215 is over lipid bilayer 214, and can hold a solution of the analyte of interest for characterization. The solution may be an aqueous solution containing bulk electrolyte 208 and buffered to an optimum ion concentration and maintained at an optimum pH to keep the nanopore 216 open. Nanopore 216 crosses lipid bilayer 214 and provides the only path for ionic flow from bulk electrolyte 208 to working electrode 202. In addition to nanopores (e.g., PNTMCs) and the analyte of interest, bulk electrolyte 208 may further include one or more of the following: lithium chloride (LiCl), sodium chloride (NaCl), potassium chloride (KCl), lithium glutamate, sodium glutamate, potassium glutamate, lithium acetate, sodium acetate, potassium acetate, calcium chloride (CaCl₂)), strontium chloride (SrCl₂), Manganese chloride (MnCl₂), and magnesium chloride (MgCl₂).

Counter electrode (CE) 210 may be an electrochemical potential sensor. In some embodiments, counter electrode 210 may be shared between a plurality of nanopore cells, and may therefore be referred to as a common electrode. In some cases, the common potential and the common electrode may be common to all nanopore cells, or at least all nanopore cells within a particular grouping. The common electrode can be configured to apply a common potential to the bulk electrolyte 208 in contact with the nanopore 216. Counter electrode 210 and working electrode 202 may be coupled to signal source 228 for providing electrical stimulus (e.g., voltage bias) across lipid bilayer 214, and may be used for sensing electrical characteristics of lipid bilayer 214 (e.g., resistance, capacitance, and ionic current flow). In some embodiments, nanopore cell 200 can also include a reference electrode 212.

In some embodiments, various checks may be made during creation of the nanopore cell as part of verification or quality control. Once a nanopore cell is created, further verification steps can be performed, e.g., to identify nanopore cells that are performing as desired (e.g., one nanopore in each cell). Such verification checks can include physical checks, voltage calibration, open channel calibration, and identification of cells with a single nanopore.

### B. Nanopore-Based Sequencing by Synthesis

Nanopore cells in nanopore sensor chip may enable parallel sequencing using a single molecule nanopore-based sequencing by synthesis (Nano-SBS) technique.

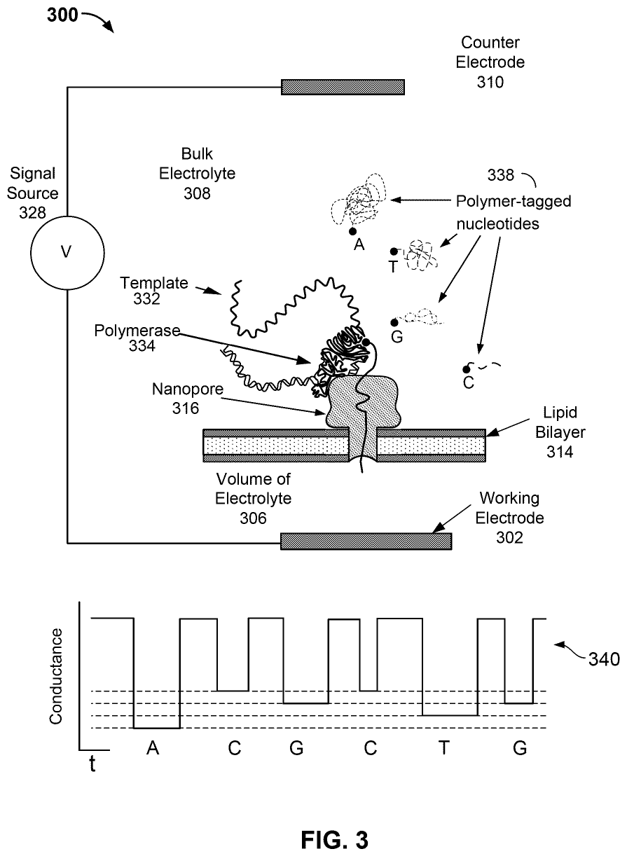

FIG. 3 illustrates an embodiment of a nanopore cell 300 performing nucleotide sequencing using the Nano-SBS technique. In the Nano-SBS technique, a template 332 to be sequenced (e.g., a nucleotide acid molecule or another analyte of interest) and a primer may be introduced into bulk electrolyte 308 in the sample chamber of nanopore cell 300. As examples, template 332 can be circular or linear. A nucleic acid primer may be hybridized to a portion of template 332 to which four differently polymer-tagged nucleotides 338 may be added.

In some embodiments, an enzyme (e.g., a polymerase 334, such as a DNA polymerase) may be associated with nanopore 316 for use in the synthesizing a complementary strand to template 332. For example, polymerase 334 may be covalently attached to nanopore 316. Polymerase 334 may catalyze the incorporation of nucleotides 338 onto the primer using a single stranded nucleic acid molecule as the template. Nucleotides 338 may comprise tag species (“tags”) with the nucleotide being one of four different types: A, T, G, or C. When a tagged nucleotide is correctly complexed with polymerase 334, the tag may be pulled (loaded) into the nanopore by an electrical force, such as a force generated in the presence of an electric field generated by a voltage applied across lipid bilayer 314 and/or nanopore 316. The tail of the tag may be positioned in the barrel of nanopore 316. The tag held in the barrel of nanopore 316 may generate a unique ionic blockade signal 340 due to the tag's distinct chemical structure and/or size, thereby electronically identifying the added base to which the tag attaches.

As used herein, a “loaded” or “threaded” tag may be one that is positioned in and/or remains in or near the nanopore for an appreciable amount of time, e.g., 0.1 millisecond (ms) to 10000 ms. In some cases, a tag is loaded in the nanopore prior to being released from the nucleotide. In some instances, the probability of a loaded tag passing through (and/or being detected by) the nanopore after being released upon a nucleotide incorporation event is suitably high, e.g., 90% to 99%.

In some embodiments, before polymerase 334 is connected to nanopore 316, the conductance of nanopore 316 may be high, such as, for example, about 300 picosiemens (300 pS). As the tag is loaded in the nanopore, a unique conductance signal (e.g., signal 340) is generated due to the tag's distinct chemical structure and/or size. For example, the conductance of the nanopore can be about 60 pS, 80 pS, 100 pS, or 120 pS, each corresponding to one of the four types of tagged nucleotides. The polymerase may then undergo an isomerization and a transphosphorylation reaction to incorporate the nucleotide into the growing nucleic acid molecule and release the tag molecule.

In some cases, some of the tagged nucleotides may not match (complementary bases) with a current position of the nucleic acid molecule (template). The tagged nucleotides that are not base-paired with the nucleic acid molecule may also pass through the nanopore. These non-paired nucleotides can be rejected by the polymerase within a time scale that is shorter than the time scale for which correctly paired nucleotides remain associated with the polymerase. Tags bound to non-paired nucleotides may pass through the nanopore quickly, and be detected for a short period of time (e.g., less than 10 ms), while tags bounded to paired nucleotides can be loaded into the nanopore and detected for a long period of time (e.g., at least 10 ms). Therefore, non-paired nucleotides may be identified by a downstream processor based at least in part on the time for which the nucleotide is detected in the nanopore.

A conductance (or equivalently the resistance) of the nanopore including the loaded (threaded) tag can be measured via a current passing through the nanopore, thereby providing an identification of the tag species and thus the nucleotide at the current position. In some embodiments, a direct current (DC) signal can be applied to the nanopore cell (e.g., so that the direction at which the tag moves through the nanopore is not reversed). However, operating a nanopore sensor for long periods of time using a direct current can change the composition of the electrode, unbalance the ion concentrations across the nanopore, and have other undesirable effects that can affect the lifetime of the nanopore cell. Applying an alternating current (AC) waveform can reduce the electro-migration to avoid these undesirable effects and have certain advantages as described below. The nucleic acid sequencing methods described herein that utilize tagged nucleotides are fully compatible with applied AC voltages, and therefore an AC waveform can be used to achieve these advantages.

The ability to re-charge the electrode during the AC detection cycle can be advantageous when sacrificial electrodes, electrodes that change molecular character in the current-carrying reactions (e.g., electrodes comprising silver), or electrodes that change molecular character in current-carrying reactions are used. An electrode may deplete during a detection cycle when a direct current signal is used. The recharging can prevent the electrode from reaching a depletion limit, such as becoming fully depleted, which can be a problem when the electrodes are small (e.g., when the electrodes are small enough to provide an array of electrodes having at least 500 electrodes per square millimeter). Electrode lifetime in some cases scales with, and is at least partly dependent on, the width of the electrode.

Suitable conditions for measuring ionic currents passing through the nanopores are known in the art and examples are provided herein. The measurement may be carried out with a voltage applied across the membrane and pore. In some embodiments, the voltage used may range from −400 mV to +400 mV. The voltage used is preferably in a range having a lower limit selected from −400 mV, −300 mV, −200 mV, −150 mV, −100 mV, −50 mV, −20 mV, and 0 mV, and an upper limit independently selected from +10 mV, +20 mV, +50 mV, +100 mV, +150 mV, +200 mV, +300 mV, and +400 mV. The voltage used may be more preferably in the range of 100 mV to 240 mV and most preferably in the range of 160 mV to 240 mV. It is possible to increase discrimination between different nucleotides by a nanopore using an increased applied potential. Sequencing nucleic acids using AC waveforms and tagged nucleotides is described in US Patent Publication No. US 2014/0134616 entitled “Nucleic Acid Sequencing Using Tags,” filed on Nov. 6, 2013, which is herein incorporated by reference in its entirety. In addition to the tagged nucleotides described in US 2014/0134616, sequencing can be performed using nucleotide analogs that lack a sugar or acyclic moiety, e.g., (S)-Glycerol nucleoside triphosphates (gNTPs) of the five common nucleobases: adenine, cytosine, guanine, uracil, and thymine (Horhota et al., Organic Letters, 8:5345-5347 [2006]).

In some implementations, additionally or alternatively, other signal values, such as electric current values may be measured and used to identify the nucleotide threaded in a nanopore.

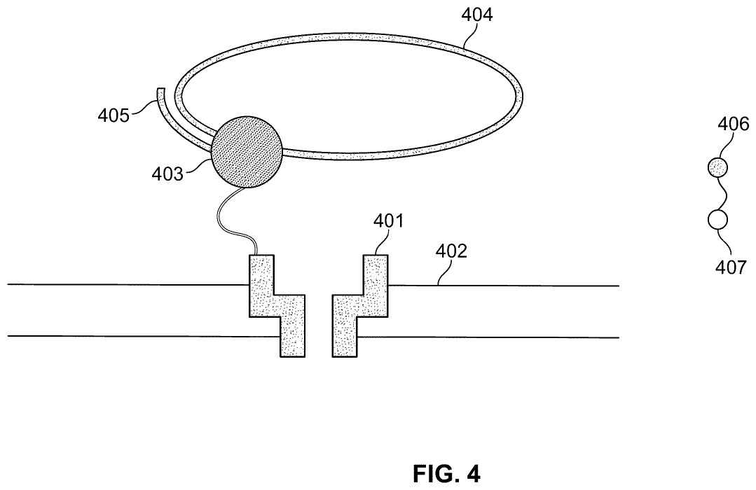

FIG. 4 illustrates an embodiment of a cell about to perform nucleotide sequencing with pre-loaded tags. A nanopore 401 is formed in a membrane 402. An enzyme (e.g., a polymerase 403, such as a DNA polymerase) is associated with the nanopore. In some cases, polymerase 403 is covalently attached to nanopore 401. Polymerase 403 is associated with a nucleic acid molecule 404 to be sequenced. In some embodiments, the nucleic acid molecule 404 is circular. In some cases, nucleic acid molecule 404 is linear. In some embodiments, a nucleic acid primer 405 is hybridized to a portion of nucleic acid molecule 404. Polymerase 403 catalyzes the incorporation of nucleotides 406 onto primer 405 using single stranded nucleic acid molecule 404 as a template. Nucleotides 406 comprise tag species (“tags”) 407.

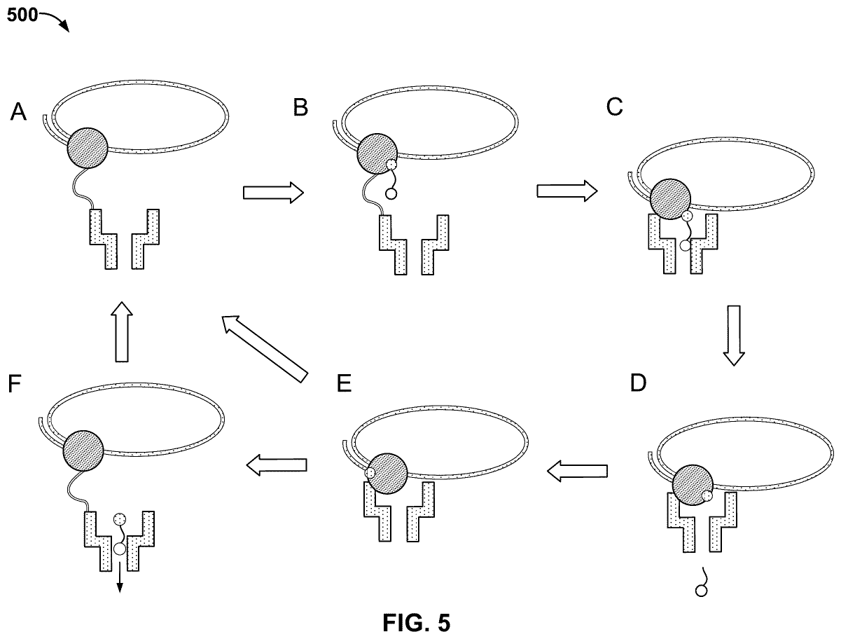

FIG. 5 illustrates an embodiment of a process 500 for nucleic acid sequencing with pre-loaded tags. Stage A illustrates the components as described in FIG. 4. Stage C shows the tag loaded into the nanopore. A “loaded” tag may be one that is positioned in and/or remains in or near the nanopore for an appreciable amount of time, e.g., 0.1 millisecond (ms) to 10000 ms. In some cases, a tag that is pre-loaded is loaded in the nanopore prior to being released from the nucleotide. In some instances, a tag is pre-loaded if the probability of the tag passing through (and/or being detected by) the nanopore after being released upon a nucleotide incorporation event is suitably high, e.g., 90% to 99%.

At stage A, a tagged nucleotide (one of four different types: A, T, G, or C) is not associated with the polymerase. At stage B, a tagged nucleotide is associated with the polymerase. At stage C, the polymerase is docked to the nanopore. The tag is pulled into the nanopore during docking by an electrical force, such as a force generated in the presence of an electric field generated by a voltage applied across the membrane and/or the nanopore.

Some of the associated tagged nucleotides are not base paired with the nucleic acid molecule. These non-paired nucleotides typically are rejected by the polymerase within a time scale that is shorter than the time scale for which correctly paired nucleotides remain associated with the polymerase. Since the non-paired nucleotides are only transiently associated with the polymerase, process 500 as shown in FIG. 5 typically does not proceed beyond stage D. For example, a non-paired nucleotide is rejected by the polymerase at stage B or shortly after the process enters stage C.

In various embodiments, before the polymerase is docked to the nanopore, the conductance of the nanopore can be ˜300 picosiemens (300 pS). As other examples, at stage C, the conductance of the nanopore can be about 60 pS, 80 pS, 100 pS, or 120 pS, corresponding to one of the four types of tagged nucleotides respectively. The polymerase undergoes an isomerization and a transphosphorylation reaction to incorporate the nucleotide into the growing nucleic acid molecule and release the tag molecule. In particular, as the tag is held in the nanopore, a unique conductance signal (e.g., see signal 310 in FIG. 3) is generated due to the tag's distinct chemical structures, thereby identifying the added base electronically. Repeating the cycle (i.e., stage A through E or stage A through F) allows for the sequencing of the nucleic acid molecule. At stage D, the released tag passes through the nanopore.

In some cases, tagged nucleotides that are not incorporated into the growing nucleic acid molecule will also pass through the nanopore, as seen in stage F of FIG. 5. The unincorporated nucleotide can be detected by the nanopore in some instances, but the method provides a means for distinguishing between an incorporated nucleotide and an unincorporated nucleotide based at least in part on the time for which the nucleotide is detected in the nanopore. Tags bound to unincorporated nucleotides pass through the nanopore quickly and are detected for a short period of time (e.g., less than 10 ms), while tags bound to incorporated nucleotides are loaded into the nanopore and detected for a long period of time (e.g., at least 10 ms).

Further details regarding the nanopore-based sequencing can be found in, for example, U.S. patent application Ser. No. 14/577,511 entitled “Nanopore-Based Sequencing With Varying Voltage Stimulus,” U.S. patent application Ser. No. 14/971,667 entitled “Nanopore-Based Sequencing With Varying Voltage Stimulus,” U.S. patent application Ser. No. 15/085,700 entitled “Non-Destructive Bilayer Monitoring Using Measurement Of Bilayer Response To Electrical Stimulus,” and U.S. patent application Ser. No. 15/085,713 entitled “Electrical Enhancement Of Bilayer Formation.”

### C. Nanopore-Based Sequencing Using Surrogate Molecules

As another example, Sequencing by eXpansion (SBX) can be used. In such a technique, the chemistry translates the sequence of DNA into a simple to measure a surrogate molecule, e.g., an Xpandomer molecule. In some implementations, Xpandomer synthesis is based on the natural function of DNA replication where expandable nucleoside triphosphates (X-NTPs) act as substrates for template-dependent, polymerase-based replication. Xpandomer synthesis can be based on four easily differentiated X-NTPs (also called High Signal-to-Noise Reporters), one for each DNA base. Engineered polymerases can incorporate these modified nucleotides into Xpandomers, exactly copying the target nucleic acid template from the library. As the Xpandomer molecule transits through the nanopore, the distinct electrical signal of each base reporter (reporter element) can be easily identifiable to enable highly accurate and high throughput nanopore-based nucleic acid sequencing.

The surrogate molecule (e.g., an Xpandomer) can be formed from a template nucleic acid molecule in the following manner. An surrogate molecule can include multiple units. Each unit can include a reporter code portion or portions (also referred to as a reporter element). The reporter codes can correspond to the different nucleotides (e.g., A. T. C, G). The reporter codes can generate different electrical signals in the nanopore and therefore allow identification of the nucleotide sequence. The surrogate molecule can be passed forward and backward through a nanopore several times to allow for multiple reads.

As some example, sequencing by expansion (SBX) using nanopores is described in WO 2020/236526 A1, “Translocation control elements, reporter codes, and further means for translocation control for use in nanopore sequencing.” filed May 14, 2020, and U.S. Pat. No. 7,939,259 B2, “High throughput nucleic acid sequencing by expansion,” filed Jun. 19, 2008, the entire contents of both of which are incorporated herein by reference for all purposes.

### II. Measurement Circuitry

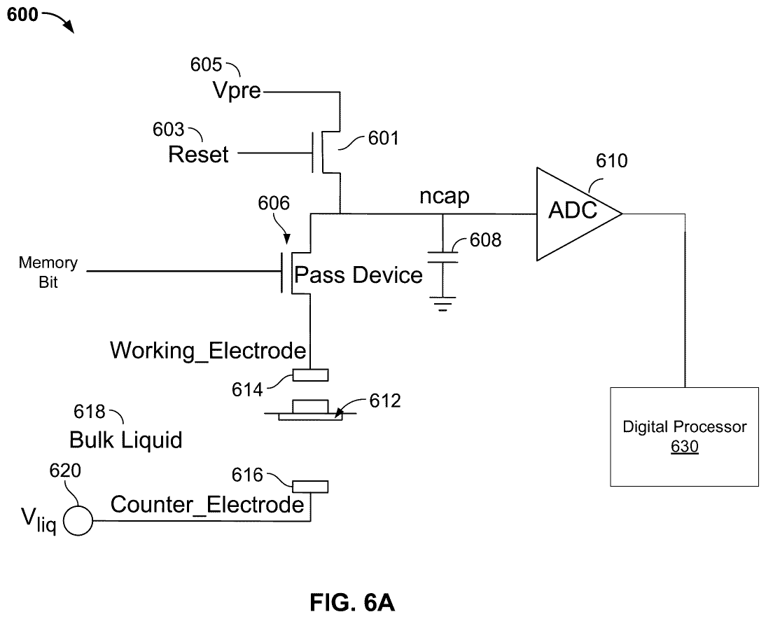

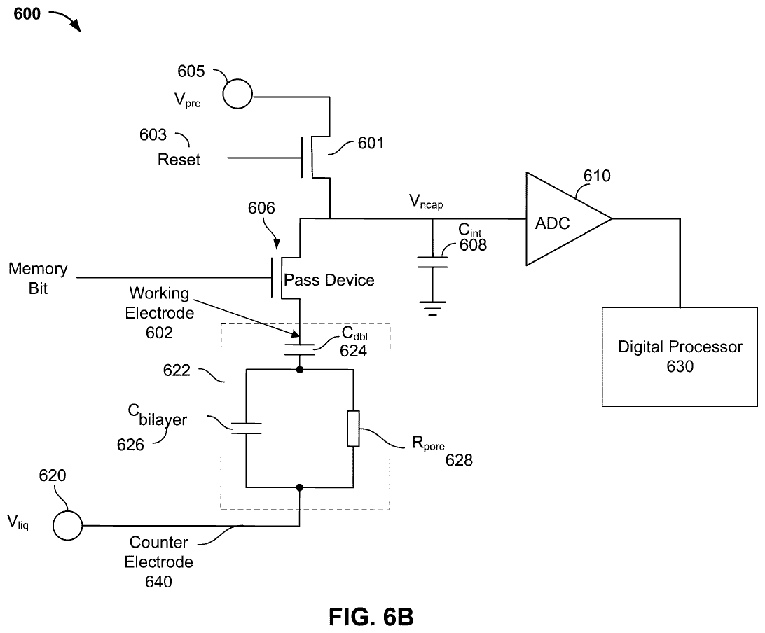

FIG. 6A shows a lipid membrane or lipid bilayer 612 situated between a cell working electrode 614 and a counter electrode 616 as part of an electric circuit 600, such that a voltage is applied across lipid membrane/bilayer 612. A lipid bilayer is a thin membrane made of two layers of lipid molecules. A lipid membrane is a membrane having a thickness of several molecules (more than two) of lipid molecules. Lipid membrane/bilayer 612 is also in contact with a bulk liquid/electrolyte 618. Note that working electrode 614, lipid membrane/bilayer 612, and counter electrode 616 are drawn upside down as compared to the working electrode, lipid bilayer, and counter electrode in FIG. 1. In some embodiments, the counter electrode is shared between a plurality of cells, and is therefore also referred to as a common electrode. The common electrode can be configured to apply a common potential to the bulk liquid in contact with the lipid membranes/bilayers in the measurements cells by connecting the common electrode to a voltage source V_{liq} 620. The common potential and the common electrode are common to all of the measurement cells. There is a working cell electrode within each measurement cell: in contrast to the common electrode, working cell working electrode 614 is configurable to apply a distinct potential that is independent from the working cell electrodes in other measurement cells.

FIG. 6B illustrates another version of electric circuit 600 in a cell of a nanopore based sequencing chip as that shown in FIG. 6A. Comparing to FIG. 6A, instead of showing a lipid membrane/bilayer between the working electrode and the counter electrode, an electrical model representing the electrical properties of the working electrode and the lipid membrane/bilayer is shown.

FIG. 6B illustrates electric circuit 600 (which may include portions of electric circuit 222 in FIG. 2) representing an electrical model in a nanopore cell, such as nanopore cell 200. As described above, in some embodiments, electric circuit 600 includes a counter electrode 640 (e.g., counter electrode 210) that may be shared between a plurality of nanopore cells or all nanopore cells in a nanopore sensor chip, and may therefore also be referred to as a common electrode. The common electrode can be configured to apply a common potential to the bulk electrolyte (e.g., bulk electrolyte 208) in contact with the lipid bilayer (e.g., lipid bilayer 214) in the nanopore cells by connecting to a voltage source V_{liq} 620. In some embodiments, an AC non-Faradaic mode may be utilized to modulate voltage V_{liq} with an AC signal (e.g., a square wave) and apply it to the bulk electrolyte in contact with the lipid bilayer in the nanopore cell. In some embodiments, V_{liq} is a square wave with a magnitude of +200-250 mV and a frequency between, for example, 25 and 600 Hz. The bulk electrolyte between counter electrode 640 and the lipid bilayer may be modeled by a large capacitor (not shown), such as 100 μF or larger.

FIG. 6B also shows an electrical model 622 representing the electrical properties of a working electrode 602 (e.g., working electrode 202) and the lipid bilayer (e.g., lipid bilayer 214). Electrical model 622 includes a capacitor C_{bilayer} 626 that models a capacitance associated with the lipid bilayer and a resistor Rₚₒᵣₑ 628 that models a variable resistance associated with the nanopore, which can change based on the presence of a particular tag in the nanopore. Electrical model 622 also includes a capacitor C_{dbl} 624 having a double-layer capacitance C_{dbl} and representing the electrical properties of working electrode 602 and the well (e.g., well 205) of the cell. Working electrode 602 may be configured to apply a distinct potential independent from the working electrodes in other nanopore cells.

Pass device 606 may be a switch that can be used to connect or disconnect the lipid bilayer and the working electrode from electric circuit 600. Pass device 606 may be controlled by a memory bit to enable or disable a voltage stimulus to be applied across the lipid bilayer in the nanopore cell. Before lipids are deposited to form the lipid bilayer, the impedance between the two electrodes may be very low because the well of the nanopore cell is not sealed, and therefore pass device 606 may be kept open to avoid a short-circuit condition. Pass device 606 may be closed after lipid solvent has been deposited to the nanopore cell to seal the well of the nanopore cell.

Electric circuit 600 may further include an on-chip integrating capacitor Cᵢₙₜ 608 (n_{cap}). Integrating capacitor Cᵢₙₜ 608 may be pre-charged by using a reset signal 603 to close switch 601, such that integrating capacitor Cᵢₙₜ 608 is connected to a voltage source Vₚᵣₑ 605. In some embodiments, voltage source Vₚᵣₑ 605 provides a constant positive voltage with a magnitude of, for example, 900 mV. When switch 601 is closed, integrating capacitor Cᵢₙₜ 608 may be pre-charged to the positive voltage level of voltage source Vₚᵣₑ 605.

After integrating capacitor Cᵢₙₜ 608 is pre-charged, reset signal 603 may be used to open switch 601 such that integrating capacitor Cᵢₙₜ 608 is disconnected from voltage source Vₚᵣₑ 605. At this point, depending on the level of voltage source V_{liq}, the potential of counter electrode 640) may be at a level higher than the potential of working electrode 602 (and integrating capacitor Cᵢₙₜ 608), or vice versa. For example, during a positive phase of a square wave from voltage source V_{liq} (e.g., the bright or dark period of the AC voltage source signal cycle), the potential of counter electrode 640 is at a level higher than the potential of working electrode 602. During a negative phase of the square wave from voltage source V_{liq} (e.g., the dark or bright period of the AC voltage source signal cycle), the potential of counter electrode 640 is at a level lower than the potential of working electrode 602. Thus, in some embodiments, integrating capacitor Cᵢₙₜ 608 may be further charged during the bright period 20) from the pre-charged voltage level of voltage source Vₚᵣₑ 605 to a higher level, and discharged during the dark period to a lower level, due to the potential difference between counter electrode 640) and working electrode 602. In other embodiments, the charging and discharging may occur in dark periods and bright periods, respectively.

Integrating capacitor Cᵢₙₜ 608 may be charged or discharged for a fixed period of time, depending on the sampling rate of an analog-to-digital converter (ADC) 610, which may be higher than 1 kHz, 5 kHz, 10 KHz, 100 kHz, or more. For example, with a sampling rate of 1 kHz, integrating capacitor Cᵢₙₜ 608 may be charged/discharged for a period of about 1 ms, and then the voltage level may be sampled and converted by ADC 610 at the end of the integration period. A particular voltage level would correspond to a particular tag species in the nanopore, and thus correspond to the nucleotide at a current position on the template.

After being sampled by ADC 610, integrating capacitor Cᵢₙₜ 608 may be pre-charged again by using reset signal 603 to close switch 601, such that integrating capacitor Cᵢₙₜ 608 is connected to voltage source Vₚᵣₑ 605 again. The steps of pre-charging integrating capacitor Cᵢₙₜ 608, waiting for a fixed period of time for integrating capacitor Cᵢₙₜ 608 to charge or discharge, and sampling and converting the voltage level of integrating capacitor by ADC 610 can be repeated in cycles throughout the sequencing process.

A digital processor 630 can process the ADC output data, e.g., for normalization, data buffering, data filtering, data compression, data reduction, event extraction, or assembling ADC output data from the array of nanopore cells into various data frames. In some embodiments, digital processor 630 can perform further downstream processing, such as base determination. Digital processor 630 can be implemented as hardware (e.g., in a GPU, FPGA, ASIC, etc.) or as a combination of hardware and software.

Accordingly, the voltage signal applied across the nanopore can be used to detect particular states of the nanopore. One of the possible states of the nanopore is an open-channel state when a tag-attached polyphosphate is absent from the barrel of the nanopore. Another four possible states of the nanopore each correspond to a state when one of the four different types of tag-attached polyphosphate nucleotides (A. T. G, or C) is held in the barrel of the nanopore. Yet another possible state of the nanopore is when the lipid bilayer is ruptured.

When the voltage level on integrating capacitor Cᵢₙₜ 608 is measured after a fixed period of time, the different states of a nanopore may result in measurements of different voltage levels. This is because the rate of the voltage decay (decrease by discharging or increase by charging) on integrating capacitor Cᵢₙₜ 608 (i.e., the steepness of the slope of a voltage on integrating capacitor Cᵢₙₜ 608 versus time plot) depends on the nanopore resistance (e.g., the resistance of resistor Rₚₒᵣₑ 628). More particularly, as the resistance associated with the nanopore in different states is different due to the molecules' (tags') distinct chemical structures, different corresponding rates of voltage decay may be observed and may be used to identify the different states of the nanopore. The voltage decay curve may be an exponential curve with an RC time constant τ=RC, where R is the resistance associated with the nanopore (i.e., Rₚₒᵣₑ 628) and C is the capacitance associated with the membrane (i.e., capacitor C_{bilayer} 626) in parallel with R. A time constant of the nanopore cell can be, for example, about 200-500 ms. The decay curve may not fit exactly to an exponential curve due to the detailed implementation of the bilayer, but the decay curve may be similar to an exponential curve and is monotonic, thus allowing detection of tags.

In some embodiments, the resistance associated with the nanopore in an open-channel state may be in the range of 100 MOhm to 20 GOhm. In some embodiments, the resistance associated with the nanopore in a state where a tag is inside the barrel of the nanopore may be within the range of 200 MOhm to 40 GOhm. In other embodiments, integrating capacitor Cᵢₙₜ 608 may be omitted, as the voltage leading to ADC 610 will still vary due to the voltage decay in electrical model 622.

The rate of the decay of the voltage on integrating capacitor Cᵢₙₜ 608 may be determined in different ways. As explained above, the rate of the voltage decay may be determined by measuring a voltage decay during a fixed time interval. For example, the voltage on integrating capacitor Cᵢₙₜ 608 may be first measured by ADC 610 at time t1, and then the voltage is measured again by ADC 610 at time t2. The voltage difference is greater when the slope of the voltage on integrating capacitor Cᵢₙₜ 608 versus time curve is steeper, and the voltage difference is smaller when the slope of the voltage curve is less steep. Thus, the voltage difference may be used as a metric for determining the rate of the decay of the voltage on integrating capacitor Cᵢₙₜ 608, and thus the state of the nanopore cell.

In other embodiments, the rate of the voltage decay can be determined by measuring a time duration that is required for a selected amount of voltage decay. For example, the time required for the voltage to drop or increase from a first voltage level V1 to a second voltage level V2 may be measured. The time required is less when the slope of the voltage vs. time curve is steeper, and the time required is greater when the slope of the voltage vs. time curve is less steep. Thus, the measured time required may be used as a metric for determining the rate of the decay of the voltage V_{ncap} on integrating capacitor Cᵢₙₜ 608, and thus the state of the nanopore cell. One skilled in the art will appreciate the various circuits that can be used to measure the resistance of the nanopore, e.g., including current measurement techniques.

In some embodiments, electric circuit 600 may not include a pass device (e.g., pass device 606) and an extra capacitor (e.g., integrating capacitor Cᵢₙₜ 608) that are fabricated on-chip, thereby facilitating the reduction in size of the nanopore-based sequencing chip. Due to the thin nature of the membrane (lipid bilayer), the capacitance associated with the membrane (e.g., capacitor C_{bilayer} 626) alone can suffice to create the required RC time constant without the need for additional on-chip capacitance. Therefore, capacitor C_{bilayer} 626 may be used as the integrating capacitor, and may be pre-charged by the voltage signal Vₚᵣₑ and subsequently be discharged or charged by the voltage signal V_{liq}. The elimination of the extra capacitor and the pass device that are otherwise fabricated on-chip in the electric circuit can significantly reduce the footprint of a single nanopore cell in the nanopore sequencing chip, thereby facilitating the scaling of the nanopore sequencing chip to include more and more cells (e.g., having millions of cells in a nanopore sequencing chip).

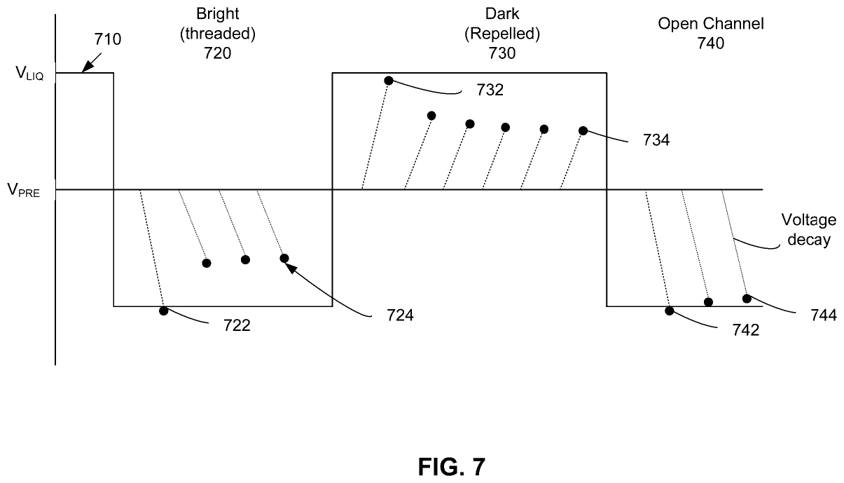

FIG. 7 shows example data points captured from a nanopore cell during bright periods and dark periods of AC cycles. In FIG. 7, the change in the data points is exaggerated for illustration purpose. The voltage (V_{PRE}) applied to the working electrode or the integrating capacitor is at a constant level, such as, for example, 900 mV. A voltage signal 510 (V_{LIQ}) applied to the counter electrode of the nanopore cells is an AC signal shown as a rectangular wave, where the duty cycle may be any suitable value, such as less than or equal to 50%, for example, about 40%.

During a bright period 720, voltage signal applied to the counter electrode by voltage source V_{liq} 620 is lower than the voltage V_{PRE} applied to the working electrode, such that a tag may be forced into the barrel of the nanopore by the electric field caused by the different voltage levels applied at the working electrode and the counter electrode (e.g., due to the charge on the tag and/or flow of the ions). When switch 601 is opened, the voltage at a node before the ADC (e.g., at an integrating capacitor) will decrease. After a voltage data point is captured (e.g., after a specified time period), switch 601 may be closed and the voltage at the measurement node will increase back to V_{PRE} again. The process can repeat to measure multiple voltage data points. In this way, multiple data points may be captured during the bright period.

As shown in FIG. 7, a first data point 722 in the bright period after a change in the sign of the V_{LIQ} signal may be lower than subsequent data points 724. This may be because there is no tag in the nanopore (open channel), and thus it has a low resistance and a high discharge rate. In some instances, first data point 722 may exceed the V_{LIQ} level as shown in FIG. 7. This may be caused by the capacitance of the bilayer coupling the signal to the on-chip capacitor. Data points 724 may be captured after a threading event has occurred, i.e., a tag is forced into the barrel of the nanopore, where the resistance of the nanopore and thus the rate of discharging of the integrating capacitor depends on the particular type of tag that is forced into the barrel of the nanopore. Data points 724 may decrease slightly for each measurement due to charge built up at C_{dbl} 624, as mentioned below.

During a dark period 730, voltage signal 710 (V_{LIQ}) applied to the counter electrode is higher than the voltage (V_{PRE}) applied to the working electrode, such that any tag would be pushed out of the barrel of the nanopore. When switch 601 is opened, the voltage at the measurement node increases because the voltage level of voltage signal 710 (V_{LIQ}) is higher than V_{PRE}. After a voltage data point is captured (e.g., after a specified time period), switch 601 may be closed and the voltage at the measurement node will decrease back to V_{PRE} again. The process can repeat to measure multiple voltage data points. Thus, multiple data points may be captured during the dark period, including a first point delta 732 and subsequent data points 734. As described above, during the dark period, any nucleotide tag is pushed out of the nanopore, and thus minimal information about any nucleotide tag is obtained, besides for use in normalization.

FIG. 7 also shows that during bright period 740, even though voltage signal 710 (V_{LIQ}) applied to the counter electrode is lower than the voltage (V_{PRE}) applied to the working electrode, no threading event occurs (open-channel). Thus, the resistance of the nanopore is low; and the rate of discharging of the integrating capacitor is high. As a result, the captured data points, including a first data point 742 and subsequent data points 744, show low voltage levels.

The voltage measured during a bright or dark period might be expected to be about the same for each measurement of a constant resistance of the nanopore (e.g., made during a bright mode of a given AC cycle while one tag is in the nanopore), but this may not be the case when charge builds up at double layer capacitor C_{dbl} 624. This charge build-up can cause the time constant of the nanopore cell to become longer. As a result, the voltage level may be shifted, thereby causing the measured value to decrease for each data point in a cycle. Thus, within a cycle, the data points may change somewhat from data point to another data point, as shown in FIG. 7.

### III. Raw Read Data Compression Architecture

In some embodiments, the sequencing system may generate raw read data at a rate greater than the capacity of one or more elements downstream from the sensors that perform the sequencing to generate raw data. The one or more elements may include elements in the data processing system being used to store or analyze the data. The one or more elements may include a channel capacity of a bus or a storage capacity. The rate difference at which data is generated and subsequently analyzed and/or stored may lead to data overload and reduce the performance of the sequencing device. Accordingly, methods and systems to compress the raw read data locally and in real-time are disclosed herein.

### A. Sequencing System

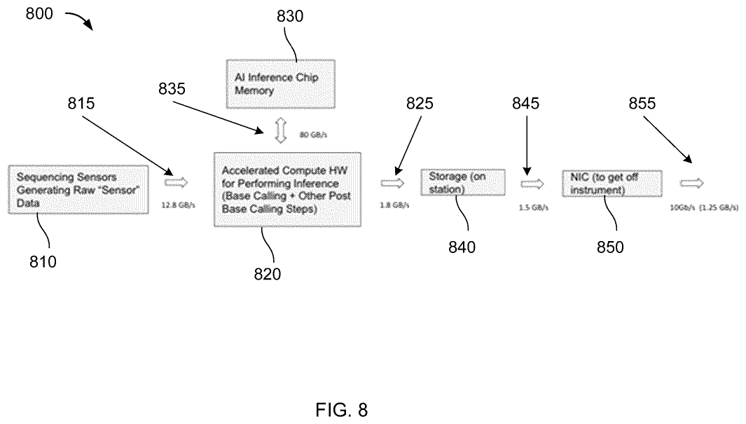

FIG. 8 shows an embodiment of a sequencing system including hardware configuration and communication channels between different components of the system. Sequencing sensors 810 generate raw data, which is then transmitted to inference circuit 820 (also referred to as an inference chip) at a rate 815. Inference circuit 820 generates a stream of raw read data comprising base calls, quality scores, and other sub-streams (e.g., header information) from the raw data. In some embodiments, rate 815 can be at least 12 gigabyte per second (GB/s).

The raw read data or sub-streams thereof, as well as the raw data and any intermediate data, can be transmitted between a memory 830 and inference circuit 820 at a rate 835. In various embodiments, the rate 835 is at least about 50 GB/s, 60 GB/s, 70 GB/s, 80 GB/s, 100 GB/s, 150 GB/s, 200 GB/s, 200 GB/s or higher. Memory 830 can buffer raw data, raw read data, or portions thereof.

The raw read data stream can be transmitted in and out of a storage device 840 at a rate 825 and 845. The storage device 840 may be an on station storage, which is a data-storage device (e.g., a hard drive or hard disk such as a solid state drive) that can be located on the same instrument as the inference chip. The rates 825 and 845 may be about 1.3-2 GB/s. In some embodiments, the rate 845 at which data is outputted from the storage device 840 (shown as on-system storage) may be lower than the input rate 825. Such rates are only examples and are used to illustrate that the downstream throughput is less than the amount of data being produced upstream, so there is a bottleneck. Various embodiments can address the bottleneck by compressing or discarding data in a particular manner that preserves accuracy.

A network inference controller (NIC) 850 can be used to offload data from storage device 840 to an external drive or disk at a rate 855. NIC can provide high transfer rates of about 1.25 GB/s (10 Gb/s). As illustrated in this example, the rate 815 at which raw data is generated is much higher than the rates at which data is transmitted to and from the storage device 840. Therefore, there is a need for compressing the data in real-time as it is generated in inference circuit 820.

As examples, inference circuit 820 can include multiple cores or chips. For instance, embodiments could have multiple GPUs (e.g., 4, 6, 8 etc.) connected by extremely high bandwidth links such as a wire-based serial multi-lane near-range communications link (e.g., NVlinks). In some instances, a dynamic random-access memory (DRAM) of one GPU can also have access to the DRAM of the next GPU.

### B. Raw Read Data Compression in Real-Time

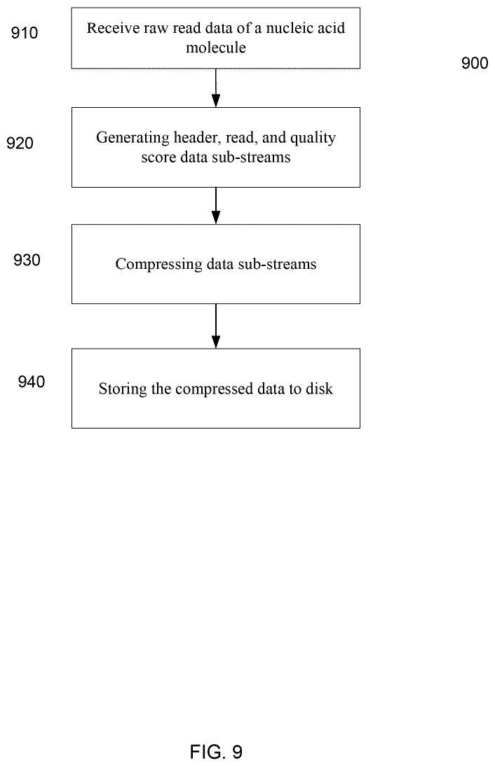

FIG. 9 is a flowchart that shows a method of real-time compression of raw read data obtained from the raw data generated by a sequencing device (e.g., nanopore-based sequencing device). The raw data may comprise sequencing data of one or more nucleic acid molecules or portions thereof. Raw read data can be generated from the raw data. The raw data can be processed by a primary analysis pipeline to generate the raw read data, for example, by an accelerated computing hardware (e.g., the inference circuit 820 in FIG. 8). The raw read data may then be stored locally (e.g., in a buffer) or provided in-real time for compression (e.g., by using the method 900). The raw data and/or the raw read data may be buffered in a memory for about 5 seconds (s), 3 s, 2 s, 1 s, 0.5 s, 0.1 s, or less. The duration of buffering the data is a small fraction of or substantially shorter than a run-cycle (e.g., the time required for the sequencing device to generate the raw data) to ensure real-time processing of the data. In some cases, the raw read data is provided for compression (e.g. by method 900) as it is generated from the raw data.

In step 910, the raw read data of a nucleic acid molecule is received (e.g., from the inference circuit 820 or memory 830). The raw read data can be received by another portion of inference circuit 820. The raw read data can be generated from the raw data by, for example, a basecalling module using the techniques disclosed in the U.S. application Ser. No. 15/669,207, which is incorporated herein by reference in its entirety and for any and all purposes.

In step 920, sub-streams, e.g., including a basecall sub-stream, a quality score sub-stream, and a header sub-stream, can be generated from the raw read data. The basecall data of the basecall sub-stream can include a sequence of basecalls for each of the plurality of nucleic acid molecules (e.g., at least 100,000 nucleic acid molecules) or portions thereof. In order to distinguish sequencing data that corresponds to separate sequencing processes or separate molecules or portions thereof, header data sub-stream may be generated. Similarly, a quality score sub-stream may be generated for each of the raw read streams. A primary analysis pipeline may convert the raw data from the sequencing device into raw read data comprising the basecall, quality score, and header sub-streams in real-time. The rate of raw read production may be on the order of about 1000 reads/see, 10,000 reads/see, 100,000 reads/see, 1,000,000 reads/see, 10,000,000 reads/see, 100,000,000 reads/see, 1,000,000,000 reads/see, or greater.

In some embodiments, the primary analysis pipeline performs step 920 in real-time. For example, the primary analysis may convert raw data from the sequencing device into raw read data as soon as the sequencing cell provides the complete raw data associated with a given sequencing cell (i.e., a given nucleic acid molecule). Alternatively, the primary analysis pipeline may perform step 920 in a quasi-real-time fashion. In some embodiments, the raw data is buffered for a period of time that may be longer than average duration of a molecular trace detection event. The raw data may be accumulated during this time, which is referred to as a time-chunk. Data of a time-chunk may be processed and all reads from a given time chunk may be generated at substantially the same time. A time chunk may last about 0.1 s. 1 s, 10 s. A time chunk may last at least about 0.1 s, 1 s, 10 s, or more. A time chunk may last at most about 10 s, 1 s, 0.1 s, or less.

In some embodiments, a portion of the raw read data can be stored temporarily. The raw read data can then be compressed at a later time. In some embodiments, the channels downstream from the sequencing device may not have the capacity to transfer, analyze, or store the raw data or the raw read data at the rate that they are produced by the sequencing device. In these cases, the raw data and/or the raw read data may be compressed before transferring or storing data.

In step 930, the raw read data stream is compressed. In some embodiments, each sub-stream in the raw read data is compressed separately. The different sub-streams in the raw read data may be analyzed and compressed simultaneously or sequentially. For example, a header sub-stream, a sub-stream of basecall data, and a quality score data sub-stream may be processed one after the other, in an ordered or unordered fashion (e.g., using multiple threads in serial, which can act as one computational thread). In some embodiments, the sub streams are compressed in parallel. Further details about compression is provided below.

In step 940, the compressed data sub-streams are transferred to a disk for storage. This may allow eliminating the need to write and/or read uncompressed data (e.g., raw data or raw read data) to or from disk. Since the raw read data is generated by the sequencing device at a very high rate, writing the high volume of raw data and/or raw read data on a disk may not be feasible due to limitations in the system, for example, limited size of available memory, I/O bandwidth, or bus channel capacity limitations. In some cases, the compressed sub-streams of raw read data are combined to generate compressed data corresponding to the sequencing data generated from the sequencing device in a single compressed data stream.

In some cases, raw read data from a time-chunk is compressed, in steps 920-930. Raw read data may also be compressed from separate time-chunks simultaneously or sequentially. The compressed data from each time-chunk may be stored in a memory (e.g., a buffer). The compressed data from separate time-chunks may then be combined into a single compressed data stream. This may be used when the data from a nucleic acid molecule is generated at different time-chunks. The combined compressed data may be stored in a memory (e.g., a buffer) so it can be merged by compressed data from the same nucleic acid molecule that are generated at later time-chunks.

### C. Read Data Sub-Stream Compression Using Separate Threads and Load Balancing

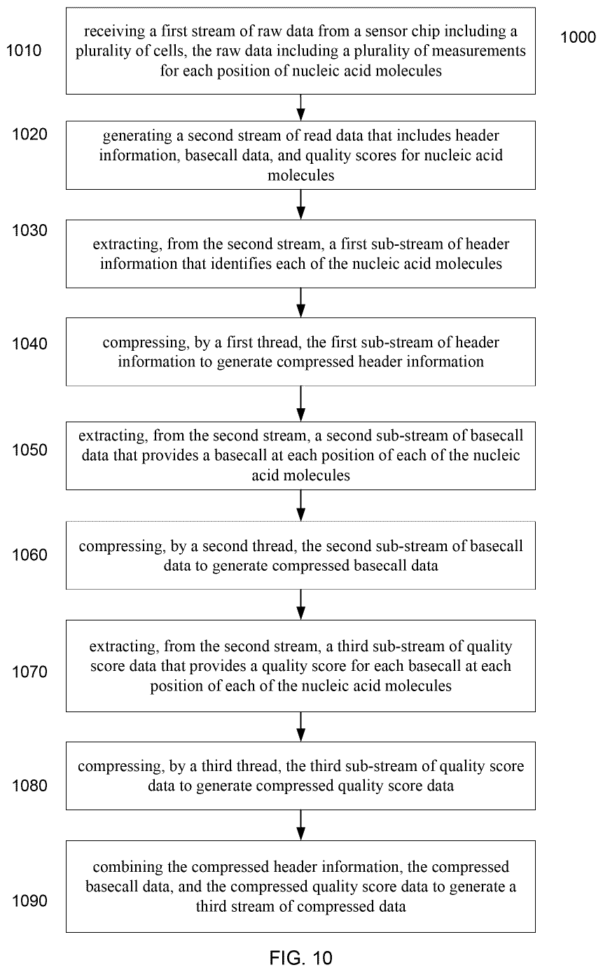

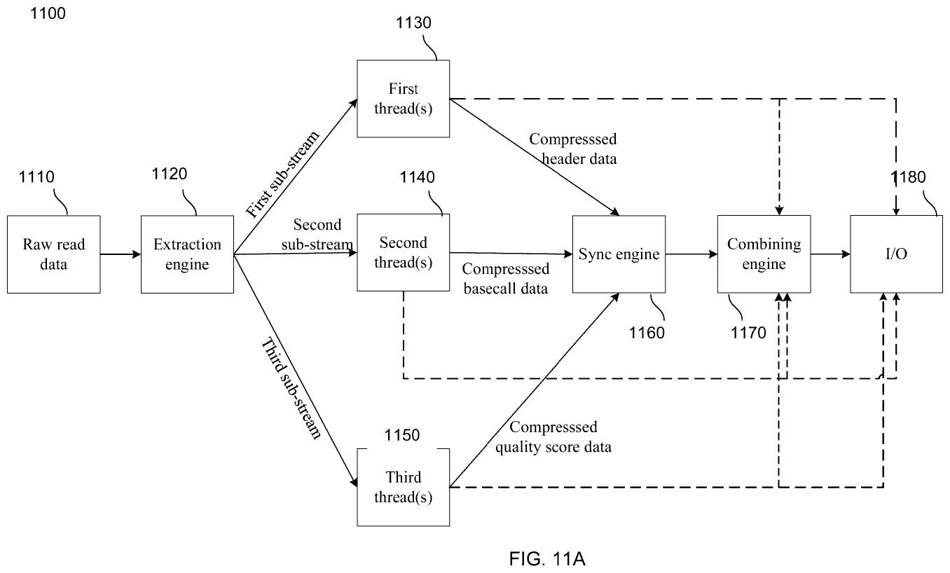

FIG. 10 is a flow chart illustrating another example method of compressing raw data generated by a sequencing device (e.g., nanopore-based sequencing device).

In step 1010, a first stream of raw data is received from a sensor chip. The raw data may include a plurality of measurements for each position of a plurality of nucleic acid molecules. The plurality of nucleic acid molecules may comprise at least 2, 3, 4, 5, 10, 50, 100, 1000, 10,000, 100,000, 500,000, one million or more nucleic acid molecules. The sensor chip may include a plurality of sequencing cells, each sequencing a separate nucleic acid molecule. In some embodiments, raw data received from the sensor chip may comprise sequencing data of multiple nucleic acids that corresponds to a same nucleic acid molecule or portions thereof. In some embodiments, raw data received from two or more of the plurality of cells in a sensor chip may comprise sequencing data that are uncorrelated to one another with respect to sequence content or their locations relative to a reference genome. For example, the raw data generated by the sensor chip from the plurality of cells may comprise sequencing information that corresponds to two or more nucleic acid molecules that may belong to different locations relative to a reference sequence.

In step 1020, a primary analysis pipeline generates a second stream of raw read data from the raw data received from the sensor chip. The raw read data can be generated from the raw data by, for example, a basecalling module using the techniques disclosed in the U.S. Patent Publication No. 2018/0037948, which is incorporated herein by reference in its entirety and for any and all purposes.

Each of the raw read data streams may correspond to one nucleic acid molecule or a particular location within the genome. In some cases, barcodes (e.g., unique or random sequence identifiers) may be attached to a nucleic acid molecule to identify the molecule. Barcodes may be attached to a nucleic acid molecule prior to sequencing. For example, unique molecular identifiers (UMIs), molecular barcodes, or random barcodes may be attached to a nucleic acid molecule or portions thereof during library preparation before the sequencing. Basecall data corresponding to such barcodes may be used to identify a nucleic acid molecule in real-time.

The second stream of raw read data, which was generated in step 1020 from raw data that corresponds to a nucleic acid molecule or a certain location on the genome, can be separated into data sub-streams. The data sub-streams may comprise a header data sub-stream, a quality score sub-stream and a basecall data sub-stream.

In step 1030, the header data sub-stream is extracted from the second stream of raw read data. The header data can have a particular format, which can be used for extracting. In other examples, particular data tags (e.g., any set of bits or characters) can be used to separate different types of data, e.g., header data from basecall data.

In step 1040, the header data sub-stream is compressed to generate compressed header information. Analyzing and compressing the header data sub-stream may be performed by one or more computational threads (threads). In some cases, the process of compressing the header data sub-stream is performed by one or more first threads. The threads may execute in parallel or in serial. As mentioned above, raw data generated by the sequencing chip may comprise sequencing information corresponding to different nucleic acid molecules or locations in the genome. The header data can contain information that identifies a read in a plurality of reads in the raw data. In some embodiments, the header data comprises strings or text. The header data can therefore be compressed as text. In some embodiments, a header data sub-stream is composed of multiple data subfields. Individual data subfields may be recognized using a data specification for each subfield. For instance, subfields can be delineated by the character length of the data or a delimiting character(s). Alternatively, the header data may be binary encoded and then compressed (e.g., lossless or lossy bit compression).

In step 1050, the basecall data sub-stream is extracted from the second stream of raw read data. The basecall data can include a sequence of basecalls for each of the plurality of nucleic acid molecules (e.g., at least 100,000 nucleic acid molecules) or portions thereof. The basecall data sub-stream comprises nucleotide type or base call for each position in the sequence read from the raw read data. The extraction can use similar techniques across the different sub-streams.

In step 1060, the basecall data sub-stream is compressed to generate compressed basecall data. In some cases, the compression of the basecall data is a lossless compression, where the entire data is substantially preserved. In other words, the lossless compression reduces the size of the data without removing a portion of the data, as opposed to lossy compression which comprises removing a portion of the data. Analyzing and compressing the basecall data sub-stream may be performed by one or more threads. The computational threads used for analyzing and compressing the basecall data sub-stream may be different from the thread(s) used to analyze and compress the header data sub-stream. In some cases, the process of compressing the basecall data sub-stream is performed by one or more second threads. The second thread may comprise one or more computational threads that may operate in parallel, sequentially, or in any combination thereof. The threads described herein may be software or hardware threads.

In step 1070, the quality score data sub-stream is extracted from the second stream of raw read data. The quality score data sub-stream comprises a probability that a base call at a given position in the sequence read is correct. The quality score may be encoded as one ASCII value (e.g., one letter).). The quality score may be encoded by converting a concrete value (e.g., a probability value between 0-1, 0-100, or 0-1000) to a discrete or categorical value (e.g., low quality, high quality, very high or very low quality, or a discrete numerical value denoting the same categories). The quality score may include multiple values for multiple features associated with each base call (multivalued features). The quality score associated with each base call may include, for example, a probability score or confidence score that a base call is correct, and a plurality of scores for the possible mismatches (e.g., comprise insertions, deletions, skips, or soft-clips) denoting the probability that the base call is a mismatch. Thus, there can be a substitution score, an insertion score, or a deletion score, or other types of scores. The features may include features other than mismatch probabilities. And, a score could be a linear combination of scores.

In step 1080, the quality score data sub-stream is compressed to generate compressed quality score data. In some cases, the compression of the quality score data is a lossy compression. Analyzing and compressing the quality score data sub-stream may be performed by one or more threads. The computational threads used for analyzing and compressing the quality score data sub-stream may be different from the thread(s) used to analyze and compress the header data or the basecall data sub-streams. In some cases, the process of compressing the quality score data sub-stream is performed by a third thread. The third thread may comprise one or more computational threads that may operate in parallel, sequentially, or in any combination thereof.

In step 1090, the compressed header data, the compressed basecall data, and the compressed quality score data can be optionally combined to generate a third stream of compressed data. In some embodiments, the compressed header data, the compressed basecall data, and the compressed quality score data are stored separately in memory (e.g., storage device, a disk, or cloud storage). Different sub-streams can be processed and compressed using separate threads.

A load balancing system can be used to manage the computational resources that are allocated to each thread. In some embodiments, the load balancing system allocates computational resources to minimize the number of computing units that are idle at any given time. This may maximize processing power and minimize processing time. In some cases, the load balancing system allocates computational resources to different thread to ensure that the compressing process of all of the sub-streams are completed almost at the same time. The computational resources may comprise computing units (e.g., CPUs, GPUs, FPGAs, memory, I/O bandwidth, etc.).

The sequence read data of the basecall data sub-stream, the header data sub-stream, and the quality score data sub-stream of one or more nucleotides may be processed and compressed at a time. The compressed data stream can be generated by adding up the compressed data for one or more nucleotides at a time. The incomplete compressed data stream can be stored in a local memory (e.g., SRAM) intermittently. The complete compressed data can then be stored in a storage device(e.g., a hard drive such as a solid state drive).

### D. Load-Balancing

Raw read data can be generated from raw data obtained from a sensor chip. A raw read data stream may comprise two or more sub-streams of basecall data, quality score data, and header data. Each of the sub-streams may comprise data that may be different (e.g., in content or format) from data of the other sub-streams. Accordingly, analyzing and compressing each sub-stream data may be performed differently (e.g., using different algorithms, threads, or different hardware). Herein, systems and methods to compress a basecall sub-stream, a quality score (q-score or Q-score) sub-stream, and a header data sub-stream are disclosed.

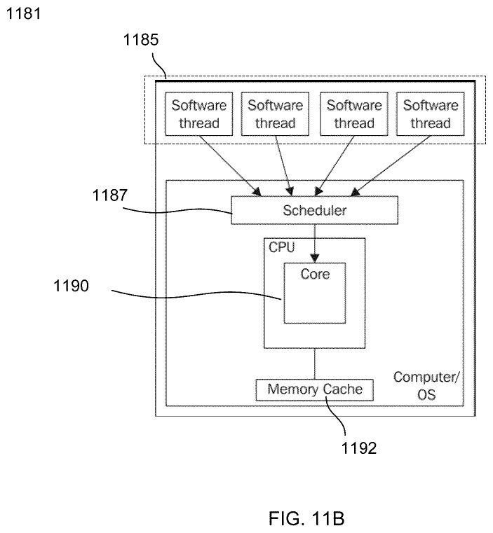

FIG. 11A illustrates an embodiment of a raw read data compression system 1100. Raw read data 1110, as mentioned hereinbefore, can be generated from raw data received from a sequencing device (e.g., by using a basecalling module). Various modules (engines) may be optional depending on the configuration used.

Sub-streams of data may then be extracted from the raw read data using an extraction engine 1120. The extraction engine 1120 may analyze the raw read data to generate a first sub-stream of header data, a second sub-stream of basecall data, and a third sub-stream of quality control data. The extraction engine 1120 may comprise logic that searches for particular characters identifying a type of data or separation markers that separate different types of data. The raw read data 1110 can be provided with portions of different types of data in a specified order, so that the next type of data after a separation marker can be pre-specified.

Each of the sub-streams may then be processed and compressed by separate computational threads. A first thread 1130 may be used to compress the first sub-stream of header data. A second thread 1140 may be used to compress the second sub-stream of basecall data. A third thread 1150 may be used to compress the third sub-stream of quality score data. In some cases, the first, the second, and the third threads may comprise one or more computational threads. In some cases, two or more sub-streams may be processed and compressed using a single thread. The first, second, and third threads may also communicate with a sync engine 1160. The threads may correspond to software threads that may be allocated to one or more processing units (e.g., time shared if allocated to a same processing unit, or executed in parallel on different processing units).

The sync engine 1160 may perform various functions. For instance, the sync engine may coordinate the scheduling of the threads. For example, sync engine 1160 can perform load balancing by assigning one or more threads to be processed by one or more processing units (e.g., CPU, GPU, FPGA, or a virtual machine). The assignment can be based on known ratios of amounts of data for the different streams, or complexity for the compression techniques (e.g., the basecalling compression requiring alignment to a reference sequence). The sync engine 1160 may receive dynamic information about a size of data being buffered for a given sub-stream, e.g., indicating that the particular sub-stream is falling behind. In such a case, sync engine 1160 can allocate more resources (e.g., time or hardware) to that sub-stream. The sync engine 1160 may also assign one or more threads to a memory unit (e.g., memory cache or buffer). The sync engine 1160 may allocate resources to the threads to ensure that sub-streams are compressed at roughly the same rate or are outputted at roughly the same time. The sync engine 1160 may then transmit the compressed sub-streams to a combining engine 1170.

In some embodiments, the hardware resources dedicated to a particular sub-stream may be dedicated (e.g., an ASIC). In such situations, sync engine 1160 can coordinate data that is output so that all the compressed data of a particular sequencing cell (e.g., a same nucleic acid) can be identified across the sub-stream, and such synced data can be sent downstream bundled together, e.g., to combining engine 1170. In other embodiments, the threads can provide the compressed data directly to combining engine 1170, and sync engine 1160 may not exist.

The combining engine 1170 can merge two or more of the compressed sub-streams to generate a single compressed data that corresponds to the raw read data 1110. In some cases, a nucleic acid molecule may be sequenced discontinuously (e.g., in time-chunks). The combining engine 1170 may comprise a buffer to store the combined compressed data from two or more raw read data (e.g., from separate time-chunks). The combining engine 1170 can then merge the combined and compressed data from different raw read data into a single compressed data. The combined and compressed data from combining engine 1170 may then be transmitted to an input-output (I/O) unit 1180. Alternatively, the compressed sub-streams may be transmitted directly to I/O 1180, e.g., when no combining is performed and instead the compressed sub-streams are output when ready. Separate chunks of each sub-stream can be buffered and output in chunks.

FIG. 11B shows an example of a load balancing system 1181 for scheduling software threads. Load balancing system 1181 may be a part of a sync engine (e.g., sync engine 1160). One or more software threads 1185 may process and compress the one or more sub-streams extracted from the raw data (e.g., using extraction engine 1120). Scheduler 1187 can allocate the one or more threads 1185 to computational processing unit 1190. Computational processing unit 1190 may comprise one or more processing units (e.g., CPU, GPU, FPGA, or a virtual machine). Scheduler 1187 may assign each thread to one or more CPUs, one or more GPUs, or combination thereof. In some cases, two or more threads may be assigned to a single processing unit (CPU, GPU, or FPGA).

Scheduler 1187 may assign the threads to processing unit 1190 based at least in part on a known ratios of amounts of data for the different threads. The assignment may be based at least in part on a dynamic information about a size of data being buffered for a given thread, e.g., indicating that the particular thread is falling behind. Scheduler 1187 may ensure that software threads 1185 are processed at roughly the same rate or are outputted at roughly the same time. Each thread may output a compressed sub-stream or a portion thereof to memory 1192. Memory 1192 may comprise one or more temporary storage units (e.g., cache memory). In some cases, outputs from one or more threads may be combined by processing unit 1190 to generate a combine compressed data or packaged into one output to be processed by a combining engine (e.g., combining engine 1170). Load balancing system 1181 may perform any of the other processes described for sync engine 1160, hereinabove.

### IV. Compression Techniques

### A. Reference Based Approach for Read Compression

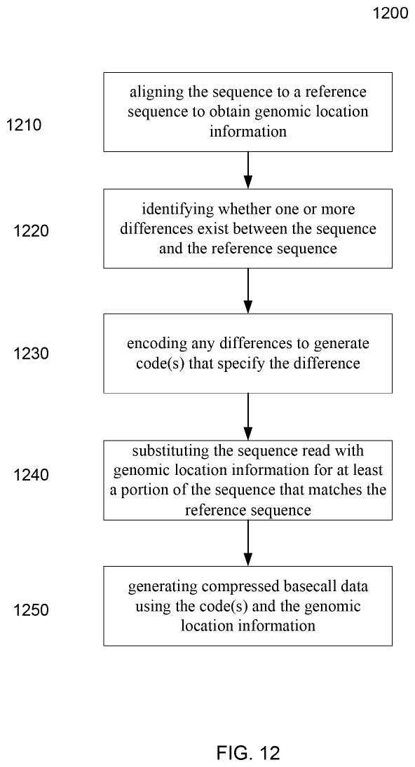

FIG. 12 is a flow chart illustrating method 1200 to compress a basecall sub-stream from the raw read data generated by a sequencing device (e.g., nanopore-based sequencing device). The basecall data can include a sequence of basecalls (also referred to as a sequence read) for each of the at least 100,000 nucleic acid molecules, or for other numbers of molecules, such as at least 2, 3, 4, 5, 10, 50, 100, 1000, 10,000, 100,000, 500,000, one million or more nucleic acid molecules. For the sequence read corresponding to a nucleic acid molecule, the basecall data comprises the base calls for each position in the sequence read. Method 1200 can be performed for each sequence of basecalls corresponding to a respective nucleic acid molecule. The compressing can be of the second sub-stream of basecall data described above.

The basecall data sub-stream stores the sequence of bases in a nucleic acid molecule (e.g., DNA or RNA), referred hereinafter as sequence read(s) or read(s). A sequence read in a basecall data sub-stream may comprise a nucleic acid sequence as a string of A, T, C, G, U or N's, where each letter denotes adenine (A), thymine (T), guanine (G), cytosine (C), uracil (U), or not-determined or ambiguous (N).

In step 1210, the sequence read is aligned relative to a reference sequence to obtain the genomic location information. This sequence alignment can be performed using various software packages, such as (but not limited to) BLAST, FASTA, Bowtie, BWA, BFAST, SHRIMP, SSAHA2, NovoAlign, and SOAP, or the techniques embodied with the software, or other techniques as known to the skilled person. The reference sequence can be a human reference sequence, such as hg18 or hg38.

The sequence alignment can generate an identifier that identifies the location within the reference sequence that the read aligns. For example, the identifier may comprise the genomic start and end locations of the reference sequence on a chromosome (e.g., a human chromosome) from the reference genome (e.g., human genome) to which the sequence read aligns. Accordingly, the alignment position relative to the reference genome may be determined. For example, the first or last aligned position of the read (e.g., closest to a 3′ or 5′ end of the reference sequence) may be used to identify the alignment position or an alignment window. Other methods may be used to store the alignment coordinates. In some cases, the read may be a positive strand or a negative strand. A read is considered “positive” strand if a read aligns without reverse complementing the sequence read. An alignment is considered “negative” strand if a sequence read is to be reverse complemented prior to alignment. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAST (e.g., BLASTn at http://www.ncbi.nlm.nih.gov/), Novoalign (Novocraft Technologies, ELAND (Illumina, San Diego, Calif.), SOAP (available at soap.genomics.org.cn), and Maq (available at maq.sourceforge.net).

In step 1220, differences between the sequence read and the reference genome are identified. The difference can be of various forms, e.g., a substitution, insertion, or deletion.

At step 1230, the outcome of the alignment including the differences identified may be used to encode the sequence read. Table 1 shows an example chart that can be used to encode a read that contains A, T, C, and Gs using 14 possible encodings. The encodings shown in Table 1 are just an example, and can be modified. The sequence read may then be encoded into a text or a bit string using the encodings. The bit string or text that is encoded at the base level can then be compressed in later steps. The encodings include a match, the 4 substitutions, 4 soft clips (the end of a read is not aligned), 4 insertions, and a deletion.

TABLE 1
Example encodings

| Char | Interpretation |
|---|---|
| = | Base matches reference |
| A | Base aligned with A substitution |
| C | Base aligned with C substitution |
| G | Base aligned with G substitution |
| T | Base aligned with T substitution |
| o | Softclip A base in read |
| p | Softclip C base in read |
| q | Softclip G base in read |
| r | Softclip T base in read |
| j | Inserted A |
| k | Inserted C |
| l | Inserted G |
| m | Inserted T |
| d | deletion |

In step 1240, the genomic location information in the reference sequence is substituted for at least a portion of the sequence that matches the reference sequence. For example, if a portion of the nucleotides in the beginning of a sequence matches with the reference sequence and then there is one or more mismatches, the nucleotides in the first portion can be replaced by a start location relative to the reference sequence, a number that shows the length of the portion, and the code that represents a mismatch. The one or more mismatches may then remain as encoded. Any portion of matching sequences may similarly be replaced (i.e., to compress the sequence data) by a start location corresponding to the position of a first matching nucleotide and a length of the portion of matching sequences. The code for a sequence match may or may not be included. A portion of the sequence that matches with a reference sequence may be 2 bases, 3 bases, 5 bases, 10 bases, 20 bases, 30 bases, 40 bases, 100 bases, 500 bases, or longer. The portion can then be substituted with, for example, only 3 numbers including a chromosome number, a start location for a location of the first nucleotide in the portion that matches with the reference sequence, and the length of the portion. In some embodiments, the length of the read must be stored as part of the location and identification of the matching bases, and may be used to decode the final compressed data.

In step 1250, compressed basecall data of the basecall data sub-stream is generated using the location information, the encoded base calls, or a combination thereof. For example, an encoded sequence read may comprise a location relative to the reference genome such as a leftmost (or rightmost) position of the read, the positions where there is a match between the read and the reference sequence, and positions where there is an insertion, a deletion, or any other encoded mismatch. To compress an encoded sequence read may then be performed by, for example, replacing the portions of the read that match the reference with the position number or a window of numbers. Different combinations of location and encoded sequence can be used to compress the sequence read.

### B. Read and Quality Score Characteristics Impacting Compression Strategies and Achievable Compression Rates

Basic characteristics of the basecall data and quality score data include the number of bits used to generate the base calls and/or the quality score (q-score) values. These basic characteristics of the basecall data and the quality score data can impact the compression rates. Table 2 shows four different scenarios, where the base calls are generated using two bits per base call with varying number of bits, from 0-6 bits, to generate each quality score value. In some embodiments, a quality score value can be generated using seven bits, six bits, four bits, three bits, two bits, one bit, or zero bit, e.g., if the quality score is not determined. The quality score may be specified using a first resolution. The quality score may be compressed by down sampling to a lower resolution. The down sampling results in a lossy compression, where at least a portion of the data may be removed in the process of compressing the data. For example, quality scores may be encoded by converting a concrete value (e.g., a probability value between 0-1, 0-100, or 0-1000) to a discrete or categorical value (e.g., low quality, high quality, very high or very low quality, or a discrete numerical value denoting the same categories). For example, a quality score of 0-1000 may be separated into four quartiles, each quartiles may then be encoded using two or more bits.

TABLE 2
Example bits for base calls and quality score (q-scores)

| | Bits per base call | Bits per q-score |
|---|---|---|
| Scenario I | 2 | 3-6 |
| Scenario II | 2 | 2 |
| Scenario III | 2 | 1 |
| Scenario IV | 2 | 0 |

### C. Examples

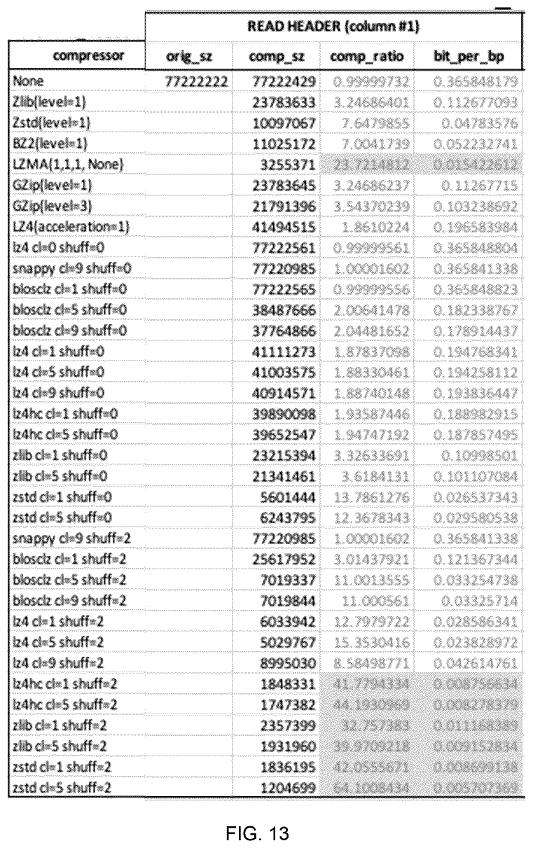

FIGS. 13-18 show results of compression rates for each separate sub-streams and the combined compressed data for a set of DNA molecules that was sequenced. Data from different sub-streams were compressed using open source compression methods. Each row represents a unique parameter combination of a compression method. The different columns in FIGS. 13-18 include “orig_siz”, “comp_sz”, “comp_ratio”, “bit_per_bp”, which respectively represent an original size of the data sub-stream before being compressed (orig_siz), a size of the data of the sub-stream after compression (comp_sz), a ratio of the original data size to the compressed data size (comp_ratio), and bits of storage per base pair of DNA read sequence (bit_per_bp), which shows a compression rate.

FIG. 13 shows results of compressing header data sub-stream. Data was compressed using various parameter combinations of eight compression methods (zlib, zstd, lzma, gzip, lz4, snappy, blosclz, lz4hc). The highest compression ratio achieved was about 64 leading to a compression rate (bit_per_bp) of about 0.006.

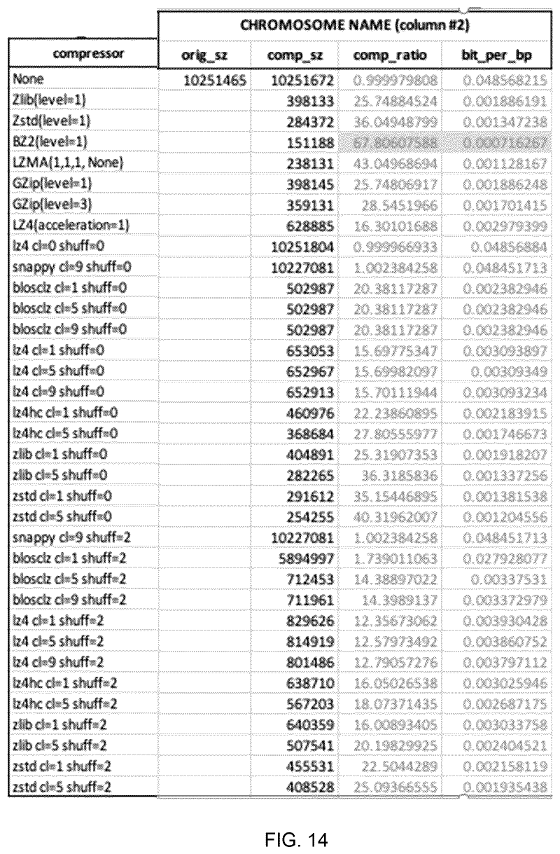

FIG. 14 shows results from compressing alignment chromosome name information. The compression algorithm achieved a compression ratio of about 70 and a compression rate of about 0.0007.

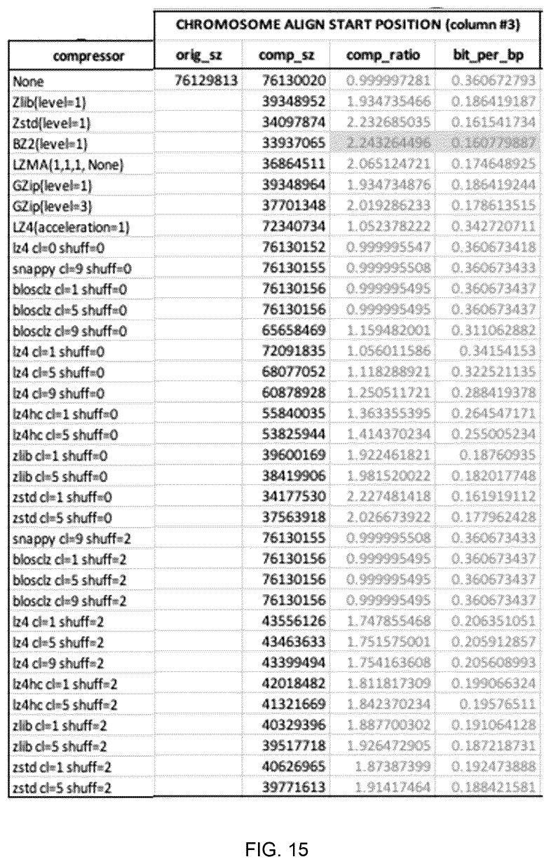

FIG. 15 shows results from compressing alignment start position information. The highest compression ratio achieved was about 2.24, which led to a compression rate of 0.16.

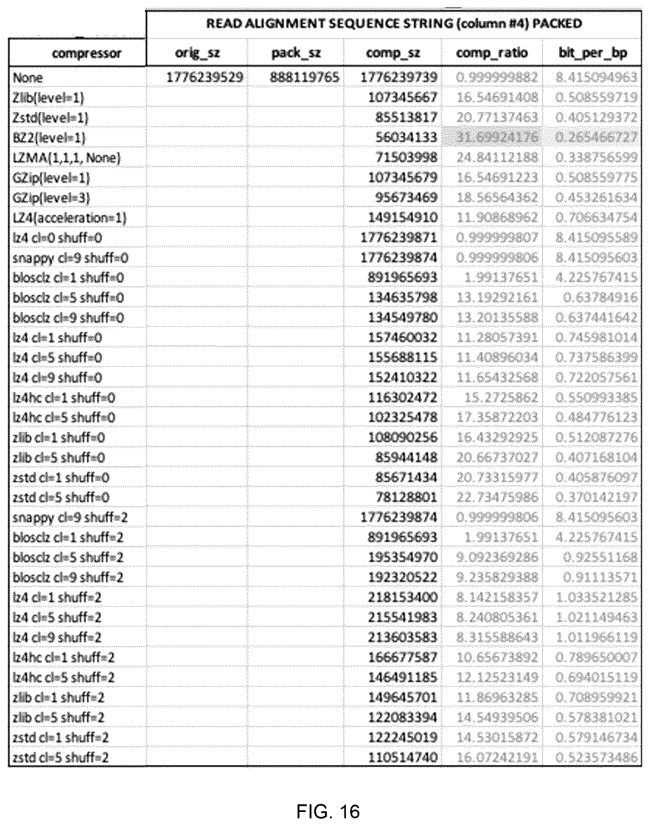

FIG. 16 shows results from compressing read sequence using a specific aligner and bit encoding. The bit encoded size of the data (pack_sz) was about half the size of the original data. The bit encoded data was then compressed using the compression methods. The highest compression ratio was about 32, which led to a compression rate (bit_per_bp) of about 0.26.

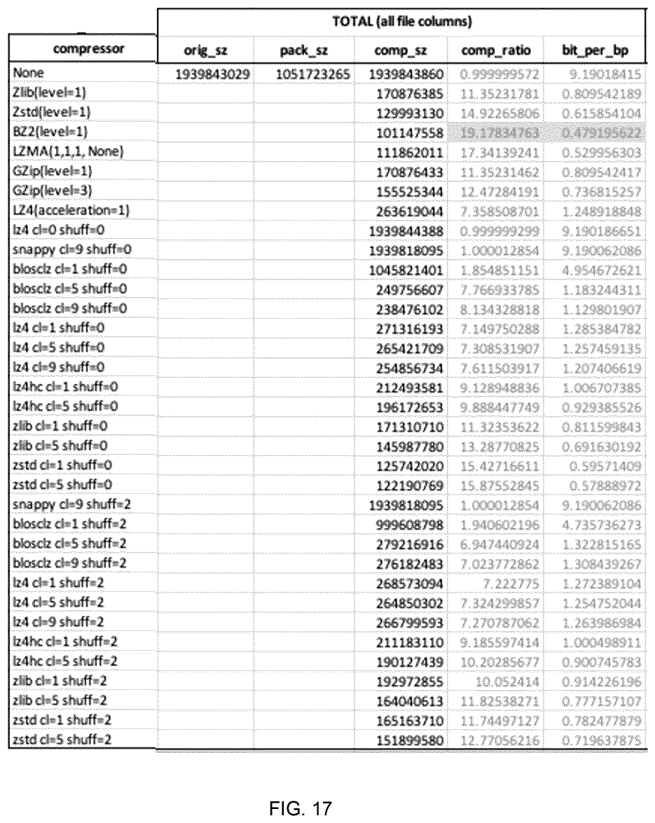

FIG. 17 shows summary results from compression.

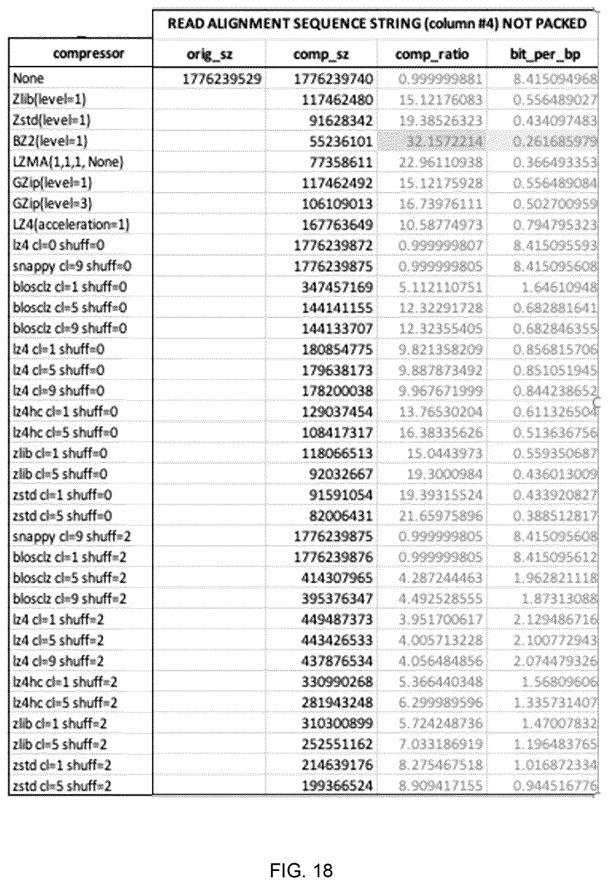

FIG. 18 shows results from compressing read sequence using a specific aligner and text encoding.

TABLE 3
Example result for compressing raw read data

| Component | Bits per base |
|---|---|
| DNA | 0.27 |
| Alignment reference id, position | 0.16 |
| and strand | |
| Quality | 0.24 |
| Read Length Required for | 0.08 |
| Decompression (assumes 16 bit | |
| INT with 200 average read length) | |
| Total | 0.75 |

The data in Table 3 is from a given configuration of a reference genome and encoding on a given dataset. These values can change based on encoding, genome (ex. Human vs. ecoli), and can change from dataset to dataset. The first row (DNA) corresponds to the number if bits needed per base in a read in the dataset after encoding relative to a reference sequence and compression of the encoded sequence. The location information (Alignment reference id, position and strand) is in the second row. The compression of the quality score requires 0.24 bits per base.

### V. Clusters, Consensus Reads, and Reducing Read Data

The higher rate of raw data generation by the sequencing device compared to the capacity of some of the channels downstream from the sensors, as described hereinbefore, may cause problems such as bottlenecks that can constrain the rate of signals, thereby limiting the throughput of the sequencing. This issue may be addressed by reducing the amount of data being transmitted through the downstream channels. The systems and methods provided herein are related to reducing the amount of sequencing data corresponding to a nucleic acid molecule in real time without negatively impacting the performance of the sequencing device (e.g., speed, accuracy, etc.). More specifically, methods and systems provided herein can be used for fast identification of a sequence read corresponding to a nucleic acid molecule or a molecular family based on an identifier (e.g., a unique molecular identifiers (UMI), a random sequence barcode (randomer), or content of a sequence read). This information may then be used in real time to discard or retain the sequence read.

An example for when a sequence read may be discarded is for clusters of reads that correspond to multiple copies of a same template nucleic acid molecule. Such clusters of sequence reads can be used to determine a consensus sequence read. But only a certain number (threshold) of sequence reads may be needed to determine the consensus sequence for the template nucleic acid. Sequence reads above the threshold can be discarded.

Accordingly, methods and systems provided herein can be used for fast identification of a sequence read corresponding to a nucleic acid molecule or molecular family based on an identifier. This information may then be used in real time to either make a decision to not save the corresponding read to disk, or to even stop sequencing a partially sequenced molecule, and clear the molecule from the sequencing device (e.g., remove the molecule form the nanopore in a nanopore-based sequencing device). Further details on clustering and bandwidth-saving techniques are described below.

### A. Barcoding the Template Molecules

Sequencing techniques are not perfect and are prone to errors in sequencing template nucleic acid molecules. Additionally, a single copy of a template nucleic acid molecule may be lost or damaged prior to or during the sequencing. Therefore, a plurality of copies of a first (template) nucleic acid molecule may be used for sequencing. The first nucleic acid molecule may be obtained from a sample (e.g., a tumor tissue sample, a liquid biopsy, or any other biological sample). The plurality of copies of the first nucleic acid molecule can be generated using amplification by, for example, polymerase chain reaction (PCR).

The first nucleic acid molecule may also be barcoded by attaching molecular barcodes to the molecule prior to amplification. Amplification of the barcoded template molecule may then generate plurality of copies of the template carrying the same barcode. A barcode may comprise a “unique molecular identifier” (UMI) sequence (e.g., a sequence used to label a population of nucleic acid molecules such that each molecule in the population has a different identifier associated with it). Barcode and UMI technologies, and methods of labeling nucleic acid molecules with a barcode or UMI sequence, are known in the art. see, e.g., Fu et al. (2014), PNAS 111:1891-1896; Islam et al. (2014) Nat Methods 11:163-168: Kivioja et al., Nat Methods 9:72-74 (2012): U.S. Pat. No. 5,604,097: U.S. Pat. No. 7,537,897: U.S. Pat. No. 8,715,967: U.S. Pat. No. 8,835,358; and WO 2013/173394.

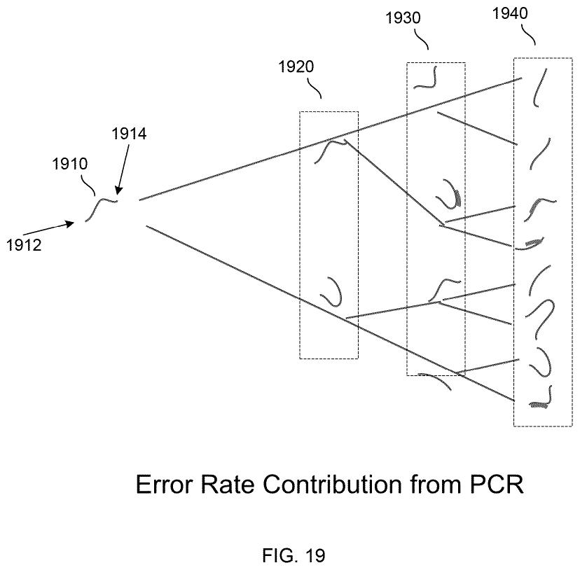

FIG. 19 illustrates an embodiment of an amplification process with molecular barcodes. A template nucleic acid molecule 1910 may be amplified to produce a first set of progeny molecules 1920, which are copies of the template nucleic acid molecule 1910. Subsequent amplification may be performed to generate more copies of the template through serial amplification. For example, a second set of progeny molecules 1930 may be amplified from progeny molecules 1920. And, a third set of progeny molecules 1940 may be generated from the progeny molecules 1940. Molecular barcodes can be attached to the template nucleic acid molecule 1910 at one or both ends 1912 and 1914. The progeny molecules 1920, 1930, 1940 may also carry the same barcode(s) as the template nucleic acid molecule 1910. A plurality of molecules including a template and its progeny molecules carrying a similar molecular barcode (e.g., random barcodes and/or UMIs) may be considered a molecular family.

The amplification may be performed using PCR. The barcode may comprise a UMI or a random sequence of nucleic acids. The barcode may be 2, 3, 4, 5, 6, 7, 8, 9, 10, 20, 30, 40, or more nucleotides long. In some cases, a barcode is at most about 50, 40, 30, 20, 10, or 5 nucleotides long. The template may be amplified for 1, 2, 3, 4, 5, 6, 7, 8, 9, 10, 50, 100, or more cycles to generate at least about 2, 4, 8, 16, 32, 64, 128, 256, 512, 1024, 2048, or more progeny molecules (i.e., amplified copies of the template).

The template and the amplified copies may then be further prepared to be sequenced via a sequencing device. In some cases, a plurality of nucleic acid molecules similar to the template may be barcoded and amplified to be processed by a sequencing device. The plurality of molecules may be obtained from one or more sample. For example, 100 molecules, 1000 molecules, 100,000 molecules, a million molecules, a billion molecules, or more may be barcoded and amplified to be processed by a sequencing device. The raw data generated from sequencing these molecules may then be processed and compressed by any of the methods and systems provided in the current disclosure comprising by encoding, using alignment techniques, clustering, or building consensus sequence reads.

### B. Clustering Sequence Reads

A population of different barcoded and amplified nucleic acid molecules may be pooled and provided to a sequencing device to be sequenced. In some cases, hundreds, thousands, millions, billions, or more barcoded and amplified molecules may be pooled to be sequenced by a sequencing device. The template molecules and copies thereof may be sequenced randomly (i.e., copies of the same molecule may be sequenced at different times or time-chunks). Raw data may be generated by a sequencing device for a population of nucleic acid molecules, at a high rate as described above and elsewhere herein. The raw data may include streams of sequence information, where each stream of raw data corresponds to a nucleic acid molecule (e.g., a barcoded nucleic acid molecule) from a molecular family.

There exist some undesirable aspects of using UMI and PCR strategy in library preparation in combination with an in silico intermolecular consensus analysis, which determines a consensus of the sequence reads all corresponding to a same template nucleic acid molecule (i.e., part of a same cluster). In some cases, the amplification and sampling process

results in uneven representation across UMI-labeled nucleic acid molecules (or UMI-molecular families). The sampling may include random sampling of the molecules generated in the amplification process. For example, a fraction of the amplified molecules (i.e., including the original template molecules) may be sampled for sequencing. Different parameters in an amplification process (e.g., number of PCR cycles) to generate different molecular families prior to sequencing may cause the molecular families to contain different number of nucleic acid molecules. This may be caused by, for example, over amplification (e.g., using PCR). Or, in some cases, an initial amount (e.g., concentration) of a nucleic acid molecule may be more than other nucleic acid molecules in a sample, leading to molecular family that contains more progenies with the same barcode and content (i.e., nucleotide sequence). Therefore, an amount of sequence reads generated by the sequencing device corresponding to a nucleic acid molecule or a molecular family may vary significantly across different molecules or molecular families. Consequently, a nucleic acid molecule or molecular family may be over-, or under-sampled. This may also happen due to other factors such as sequencing errors.

This may be undesirable from an assay perspective. For example, if a particular assay has some desired depth of coverage for each UMI-molecular family (e.g., 10×), the resulting intermolecular consensus families (clusters) may hit that average 10x read depth, but the variance across families will be high. Thus some molecular families may have insufficient representation, while others may have orders of magnitude more reads than are required. Families with extremely high depth of coverage may not benefit the assay much, while the UMI-molecular families with membership number lower than the desired depth will be unable to generate high quality consensus reads. For example, each family labeled using a UMI may represent a region of interest in a genome. In order to satisfy assay needs for all regions of interest, the sequencing throughput requirements has to be raised in order for all regions of interest to be covered by at least the minimum required depth. The regions of interest can be the subject of targeted sequencing. e.g., enrichment of DNA from those regions, as may be done by amplification of DNA or capture probes.

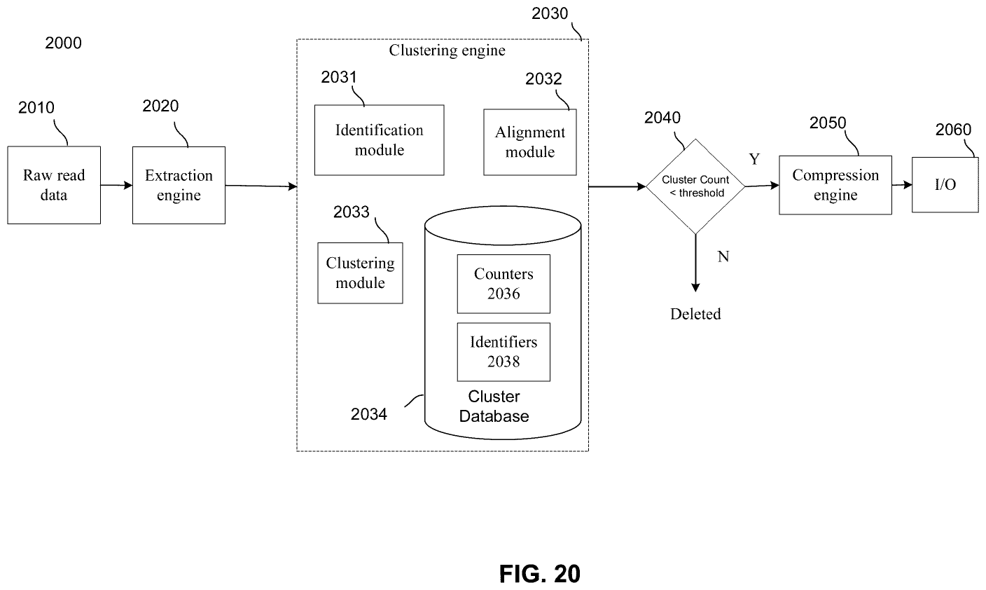

FIG. 20 illustrates an embodiment of sequence read data clustering system 2000. Raw read data is received as input 2010. The raw read data can be generated by an inference circuit from raw data received from the sequencing device (i.e., a sensor chip including a plurality of cells), as described above or elsewhere herein. The raw read data may then be transmitted to an extraction engine 2020, where basecall data comprising nucleotide information for each positon in a sequence read of a template molecule is extracted from the raw read data. The basecall data may then be processed by a clustering engine 2030, more details of which are described herein below.

The clustering engine 2030 may determine cluster information by comprising a size of a cluster to a cluster count module 2040. The size of a cluster can correspond to a current count of reads assigned to the cluster. The data comprising the raw read data may then be transmitted to a compression engine 2050 or be discarded based on the comparison made by the cluster count module 2040. If the size has already exceeded a threshold, then any further reads can be discarded. The read data that is transmitted to the compression engine may then be processed and compressed using any of the methods described herein and sent to an I/O 2060.

The clustering engine 2030 may comprise a barcode module 2031, an alignment module 2032, and a clustering module 2033. The clustering engine 2030 may also include or may have access to a cluster database 2034. The barcode module 2031 can identify a barcode sequence in a sequence read. Alignment module 2032 may perform sequence alignment between a sequence read and sequence corresponding to a cluster or a reference sequence. The sequence read may then be assigned to a cluster by clustering module 2033 based at least partially on the output from alignment module 2032 (e.g., a sequence similarity or a read location relative to a reference sequence.) The clustering module 2033 can cluster sequence reads, where each cluster contains sequence reads corresponding to a same template nucleic acid molecule or molecular family.

The cluster database 2034 may include information corresponding with each of the clusters, so as to determine whether a new read belongs to an existing cluster or whether a new cluster should be created. This information may be stored in the cluster database 2034 in identifiers 2038. Identifiers 2038 may comprise information corresponding to a barcode information and/or a location information of one or more sequence reads that are assigned to a cluster (e.g., start and/or end position relative to a reference sequence). The identifiers of a cluster may also comprise a sequence read content (e.g., of another sequence read in the cluster or a consensus read of all the reads in a cluster). For example, a start and/or stop coordinates of a sequence read may be used as an identifier or a portion thereof. In some cases where a consensus is determined on the inference circuit, a consensus sequence can be generated for each cluster incrementally as each sequence read is assigned to the cluster. In such cases, for each cluster the consensus sequence or its location can be stored in identifiers 2038.

The number of sequence reads assigned to a cluster can be stored in the cluster database 2034 as a counter value for that cluster in counters 2036. The counter value for each particular cluster may increase incrementally as a new sequence read is assigned to that particular cluster. The information in cluster database 2034 may be accessed by the different modules in the search engine (i.e., 2031, 2032 and 2033).

The clustering module 2033 may assign a sequence read to a cluster based on the output from the barcode module 2031 and/or the alignment module 2032, along with the information in identifiers 2038. Therefore a sequence read may be assigned to a cluster by comparing the sequence or its location (e.g., relative to a reference sequence) with identifiers 2038 to determine a match.

A barcode may comprise a random sequence barcode, a UMI, or a combination thereof. The barcode module 2031 can identify the barcode sequence in a sequence read in real time. The barcode module 2031 may then compare (e.g., by sequence alignment) the barcode sequence of a sequence read to barcode sequences corresponding to different clusters (e.g., from the identifiers 2038 in the cluster database 2034). The barcode module 2031 can also compare barcode sequences of one or more sequence reads to one another to assign them to different clusters. For example, in cases where a particular barcode sequence of a sequence read is not present in the cluster database 2034 (i.e., a nucleic acid molecule with a particular barcode has not been sequenced prior). In some cases, clustering module 2033 assigns sequence reads to different clusters partially based on the barcode module 2031.

Sequence reads may be analyzed using the alignment module 2032. The alignment module 2032 can align a sequence read to a reference sequence and/or to one or more other sequence reads. An output of alignment module 2032 may be used in addition to (or independent from) an output from barcode module 2031 to cluster new sequence reads (e.g., by the clustering module 2033). For a particular sequence read, if the alignment module 2032 does not find a similar sequence (e.g., by comparing the sequence content or location relative to a reference sequence) in any of the existing clusters, the clustering module 2033 may assign the sequence read to a new cluster.

In one example, alignment module 2032 may align the sequence read to a reference sequence (e.g., of a reference genome), the alignment module 2032 may then determine a location of the sequence read relative to a reference sequence. The location of the sequence read may then be compared to the location of sequences of a cluster to identify a cluster corresponding to the sequence read.

In another example, alignment module 2032 may align the sequence read to a sequence read already assigned to a cluster representing that cluster. Alternatively, alignment module 2032 may comprise a multiple sequence alignment algorithm. The sequence read may then be aligned with two or more of the sequence reads (or all of the sequence reads) in a cluster via the multiple sequence alignment algorithm. A sequence similarity criterion (e.g., a minimum similarity) may be considered to assign the sequence read to a cluster. The sequence read may be assigned to the cluster that leads to the highest sequence similarity when aligned to the sequence read.

In yet another example, alignment module 2032 may align the sequence read to a consensus sequence representing the sequences of a cluster. The consensus sequence may be generated for each cluster incrementally as new sequence reads are assigned to each cluster. A sequence similarity criterion (e.g., a minimum similarity) may be applied to the output of the alignment to assign the sequence read to a cluster. The sequence read may be assigned to the cluster with a consensus that produced the highest sequence similarity when aligned to the sequence read.

In some embodiments, a consensus read for a cluster can be used as a reference against which all the reads in the cluster could be compressed. For example, assume there are 100 reads in a cluster, with each read ˜350 bp long and there is a true deletion in the sample, where the deletion shows up in almost all of those reads. Then, instead of performing a delta compression of each read against the reference independently, the consensus read can be stored with the deletion relative to the reference. Then, for compressing each of the read, the reads can be mapped to the consensus read and delta compression performed against the consensus. This may result in a higher compression ratio for the reads in that cluster.

Optimal alignment by alignment module 2032 may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAST (e.g., BLASTn at http://www.ncbi.nlm.nih.gov/), Novoalign (Novocraft Technologies, ELAND (Illumina, San Diego, Calif.), SOAP (available at soap.genomics.org.cn), and Maq (available at maq.sourceforge.net). Two or more sequence reads may have the same content if they have a medium, high, or very high sequence similarity. In some cases, two or more sequences having a same content may have a sequence similarity of at least about 70%, 80%, 90%, 95%, 99%, or more. In some cases, two or more sequence reads are considered the same when they have a sequence similarity of at least 94%.

In the absence of a barcode or when the barcode(s) match two or more clusters, clustering may be performed using the output from the alignment module 2032. For example, alignment module 2032 may align the new sequence read to a sequence corresponding to a cluster with similar barcodes. The output may be used to assign the sequence read to a cluster or create a new cluster, e.g. in a clustering of a set of sequence reads. If the sequence reads cannot be assigned to existing clusters, the output from clustering module 2033 can be used by clustering module 2033 to generate new clusters using clustering algorithms. Some clustering algorithms use single-linkage clustering, constructing a transitive closure of sequences with a similarity over a particular threshold. Examples of these algorithms include BLASTClust (nih.gov) and CluSTr (ebi.ac.uk/clustr). UCLUST (drive5.com/usearch) and CD-HIT (cd-hit.org) use a greedy algorithm that identifies a representative sequence for each cluster and assigns a new sequence to that cluster if it is sufficiently similar to the representative: if a sequence is not matched then it becomes the representative sequence for a new cluster. The similarity score is often based on sequence alignment. Sequence clustering is often used to make a non-redundant set of representative sequences.

### C. Discarding Over-Represented Data

In order to balance the amount of sequence reads across different molecules, sequence reads that are clustered using the clustering engine 2030 may be counted for each cluster. Each cluster may correspond to a nucleic acid molecule or a molecular family. A cluster may comprise one or more sequence reads corresponding to a same nucleic acid molecule or molecular family. A size of a cluster (i.e., the number of sequence reads assigned to a cluster) may be controlled to reduce over-representation in one or more clusters compared to other clusters. The size of a cluster may be monitored by a counter as described herein above. As the clustering module 2033 assigns a sequence read to a particular cluster, a counter may increment the size of that cluster.

The size of a cluster may be controlled to reduce the amount of data (e.g., sequence read data corresponding to a nucleic acid molecule or molecular family) that may be stored in a memory and/or to be transmitted out (e.g., to a storage device) to reduce constrains produced by bottlenecks. In some cases, a threshold may be applied to control the cluster size. The output from the clustering engine 2030 may be provided to a cluster count module 2040. The output from the clustering engine may comprise the sequence read data (or basecall data) and the cluster information (e.g., cluster identification and counter value) that the sequence read is assigned to. The cluster count check may compare the counter value in the cluster information with the threshold value. If a counter for a particular cluster exceeds the threshold, a new sequence read that is assigned to that particular cluster may be discarded from the system. Alternatively, a sequencing procedure for a partially sequenced molecule associated with the new sequence read may be stopped, and the corresponding nucleic acid molecule may be cleared from the sequencing device (e.g., by removing the nucleic acid molecule form the nanopore in a nanopore-based sequencing device). If the cluster count value is below the threshold the cluster count module 2040 may transmit the output received from the clustering engine 2030 to a downstream module.

In some cases, the cluster count module 2040 transmits data to a compression engine 2050 to process and compress the data using any of the methods described above or elsewhere herein. In some cases, the compression engine (e.g., using techniques described herein, such as in section IV) may process the sequence read data to generate a consensus sequence read for the cluster corresponding to a nucleic acid molecule or molecular family. Alternatively, the cluster count module 2040 may transmit the data directly to an input/output (I/O) 2060, for example, to be stored in a storage device. Reducing data as described above (i.e., pruning data) and elsewhere herein, can improve the performance of the computer as well as the sequencing device as it improves memory usage and reduces the constraints imposed on the system by bottlenecks (e.g., bus capacity and I/O rates that are lower than raw data generation by the sensor chips).

### D. Flowchart

Methods and systems provided herein comprising clustering and building consensus reads can be used to mitigate the over-sampling issue and also reduce the amount of data that needs to be stored for each nucleic acid molecule or molecular family in order to generate accurate nucleotide sequence of each of the nucleic acid molecules.

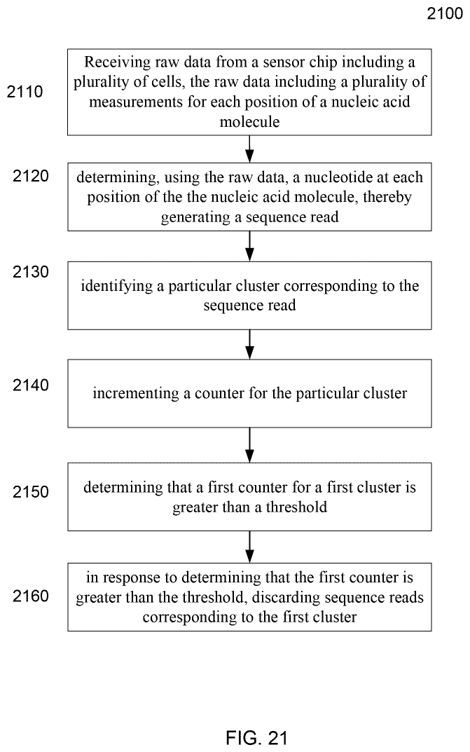

FIG. 21 shows a flow chart of method 2100 for clustering sequence reads to reduce an amount of sequencing data according to embodiments of the present disclosure.

In step 2110, raw data is received from a sensor chip. The raw data may include a plurality of measurements for each position of a respective of nucleic acid molecule of a plurality of nucleic acid molecules. The plurality of nucleic acid molecules may comprise at least 2, 3, 4, 5, 10, 50, 100, 1000, 10,000, 100,000, or more nucleic acid molecules. The sensor chip may include a plurality of sequencing cells, each sequencing one or more separate nucleic acid molecules. At least a portion of the plurality of nucleic acid molecules (e.g., at least 100,000 nucleic acid molecules) can include clusters of nucleic acid molecules. The nucleic acid molecules of a cluster may correspond to a same template nucleic acid molecule.

In step 2120, for each position of a respective nucleic acid molecule, using the raw data, a nucleotide at the position may be determined, thereby generating a sequence read for the respective nucleic acid molecule. In some cases, a template is barcoded (e.g., using a unique molecular identifier (UMI), or a random identifier (randomer)). The sequence read of a barcoded template may then comprise the sequence of the barcode and well as the sequence information of the nucleic acid sequence. The barcode may comprise one or more barcodes including UMIs, randomers, or a combination thereof.

In step 2130, for each sequence read for the plurality of nucleic acid molecules (e.g., at least 100,000 nucleic acid molecules), a particular cluster may be identified. The cluster may correspond to the sequence read. A particular barcode may be assigned to the particular cluster (e.g., when a barcode is unique such as a UMI). In some cases, a particular cluster may correspond to one or more particular barcode sequences. A particular cluster corresponding to a sequence read may be identified by comparing one or more barcode sequences of the sequence read to the one or more particular barcode sequences that a particular cluster corresponds to. If a match is determined the sequence read may be assigned to the particular cluster. If one or more barcode sequences of the sequence read do not match to the one or more particular barcode sequences assigned to existing clusters, a new cluster may be created corresponding to the sequence read.

Identifying a particular cluster corresponding to a sequence read may include comparing a genomic location of the particular cluster with the genomic location of the sequence read. A genomic location may be determined by aligning a sequence (e.g., a sequence read, or a sequence that a particular cluster corresponds to) to a reference sequence. The genomic location may include a start genomic location and an end genomic location relative to the reference sequence. The genomic location of the particular cluster may correspond to a genomic location of a sequence read that has already been assigned to that particular cluster.

In some cases, two or more clusters may be assigned the same barcode (e.g., a randomer). The sequence information of the nucleic acid sequence that are assigned to the one or more clusters can then be compared. The sequence information of the nucleic acid sequence that are assigned to the one or more clusters may be different from one another. In other word, unique sequence reads comprising the information of the nucleic acid sequence and the randomer may be assigned to each cluster. Where, each unique sequence read correspond to a different template nucleic acid molecule. A cluster may then be generated by making copies of a template nucleic acid. The copies may be generated using polymerase chain reaction (PCR).

In step 2140, a counter for the particular cluster may be incremented as for each sequence read a particular cluster is identified. A counter may record the number of sequence reads that are assigned to a particular cluster.

In step 2150, a first counter for a first cluster may be compared to a threshold to determine if the first counter is greater than the threshold. The threshold may be predetermined (e.g., provided by a user). The threshold may be calculated based on one or more factors including a length of the sequence read, nucleic acid content of the sequence read (e.g., A, T, C, G, or U bases) an error rate associate with sequencing, amplification (e.g., PCR), and/or barcoding. The threshold may be about 10, 20, 30, 40, 50, 60, or more.

In step 2160, in response to determining that the first counter is greater than the threshold, the sequence read corresponding to the first cluster may be discarded. If the number of sequence reads that are assigned to the first cluster is smaller than the threshold, the sequence reads may remain associated with the cluster (i.e., remain stored in a memory). The sequence reads corresponding to a cluster may be output (e.g., from the inference circuit), when the counter is less than or equal to the threshold. The sequence read assigned to the first cluster with a first counter that is equal or greater than the threshold, may be discarded. Limiting the number of sequence reads assigned to a cluster may reduce the amount of data that may be stored or transmitted out of the sequencing system. Accordingly, this may reduce the constrains produced by bottlenecks in the system, as described before or elsewhere herein.

### E. Forming Intermolecular Consensus Read for Each Cluster

As mentioned above, each cluster may contain a plurality of sequence reads that correspond to a nucleic acid molecule. In order to reduce the amount of data within a cluster, sequence reads may be collapsed into a single sequence read representing a consensus sequence. This consensus is an intermolecular consensus as sequence reads from multiple nucleic acid molecule are used. An intramolecular consensus determined from a single nucleic acid molecule is described in the next section. The consensus sequence of a cluster is a single nucleotide sequence, in which every position is a nucleotide that is most commonly called amongst all the sequence reads in that cluster. The consensus sequence may be generated by performing a multiple alignment between all the sequence reads in a cluster. Alternatively, the consensus sequence may be generated by aligning each sequence read in a cluster to a reference genome. Then, for every position in the multiple alignment or alignment to a reference genome, the most common nucleotide amongst all reads can be selected.

Each sequence read may contain random errors that can be randomly produced during nucleic acid amplification and sequencing processes. A consensus sequence, generated from a plurality off sequence reads, may therefore more accurately represent a nucleic acid molecule. Including more sequence reads to form a consensus sequence read may lead to a consensus sequence read that may correspond to the actual sequence of the nucleic acid molecule more accurately. On the other hand, including too many sequence reads to generate a consensus read may consume more time as well as more memory, and computational resources. Therefore to optimize generating an accurate consensus data, a cutoff can be applied to a number of sequence reads that are used in building the consensus. For example, a highly accurate consensus sequence may be generated from at most about 100, 50, 40, 30, 20, 10, or less sequence reads.

A threshold data for a size of cluster may directly correspond to this cutoff value. In some cases, the threshold for a size of cluster may be based at least in part on this cutoff value. In some cases, the threshold for a size of cluster may be the same as this cutoff value. For example, a consensus read corresponding to a nucleic acid sequence is generated using only a number of sequence reads that is equivalent or less than the cutoff value. Any sequence read that corresponds to a nucleic acid molecule that has a number of sequence reads that exceed a cutoff value may be discarded from the system (e.g., deleted from the memory). In some cases, a consensus read may generated at the time of transmission to a downstream module or an I/O as soon as the number of sequence reads reaches the cutoff value for a nucleic acid molecule.

In some cases, a second cutoff value may be used to ensure a high quality in consensus reads. The second cutoff value may comprise a lower limit for the number of sequence reads used to generate a consensus sequence. In some cases, at least 2, 3, 5, 10, 20, 30, 40, 50, 60, or more sequence reads are used to build the consensus sequence. For example, a consensus read may not be generated or be output unless a number of sequence reads corresponding to a nucleic acid molecule that exceeds a second cutoff is provided. In some cases, a message can be generated to show that the number of sequence reads that correspond to a nucleic acid molecule is not enough to generate a consensus read.

### F. Intramolecular Consensus

In some embodiments, a nucleic acid molecule may be sequenced multiple times, thereby providing multiple sequence reads (also called subreads). For example, the molecule can be passed back and forth within a nanopore, with each pass providing a sequence read. In such an example, an intramolecular consensus can be created. The intramolecular consensus can be determined at each position based on the majority base call at that position across the individual subreads. The multiple passes can provide a more accurate final read (intramolecular consensus) than any one of the individual subreads.

As described in FIG. 19, each of the progeny molecules 1940 are sequenced. An xpandomer molecule can be generated for each of these progeny molecules 1940. The xpandomer molecule can be passed multiple times through a nanopore, thereby providing multiple sequence reads. An intramolecular consensus can then be determined. The intramolecular consensus for each progeny molecule can then be used to determine the intermolecular consensus.

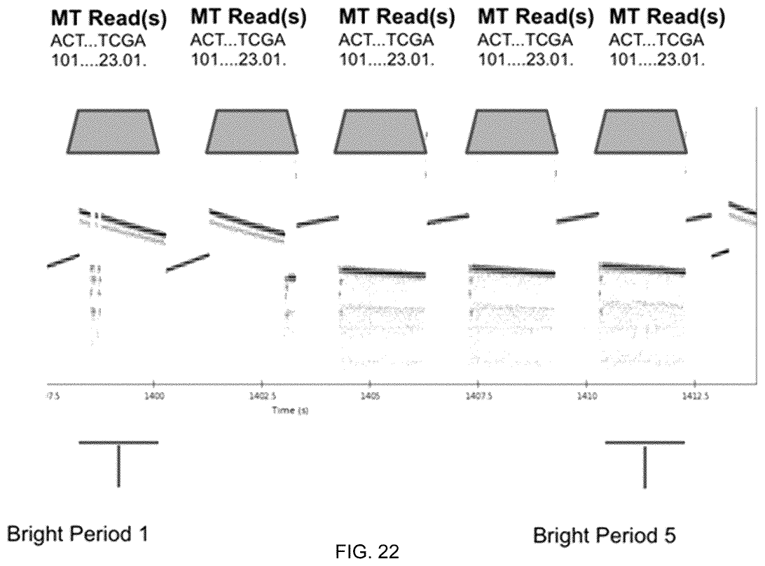

FIG. 22 shows the raw data for multiple passes of an xpandomer molecule being read using a nanopore. The xpandomer molecule can be trapped in a nanopore for reading the same molecule multiple times. An example of a “trapped” molecule is indicated in the raw trace in FIG. 22, where a single xpandomer has been trapped in periods 2, 3, 4, 5. In this scenario the same molecule is read 4 more times, and these subreads from the same molecule occur proximally in time. This naturally clustering of reads in time, presents an advantage for the formation of consensus reads.

From a data movement perspective, one draw back of intermolecular consensus is that it is not easily amenable to online processing, or is at least more difficult to perform in an online fashion. Reads corresponding to membership in the same molecular family are spread out randomly in time over the course of a run. Therefore, given a lack of predetermined position in time for read members of individual molecular families, it is easier to wait until the end of a run to begin the read clustering step needed for consensus. The approach of trapped molecules circumvents this problem. Since the subreads are known to be sequential in time, the consensus can be determined at that time, and just the consensus can be passed to the next stage. The reads themselves can be discarded.

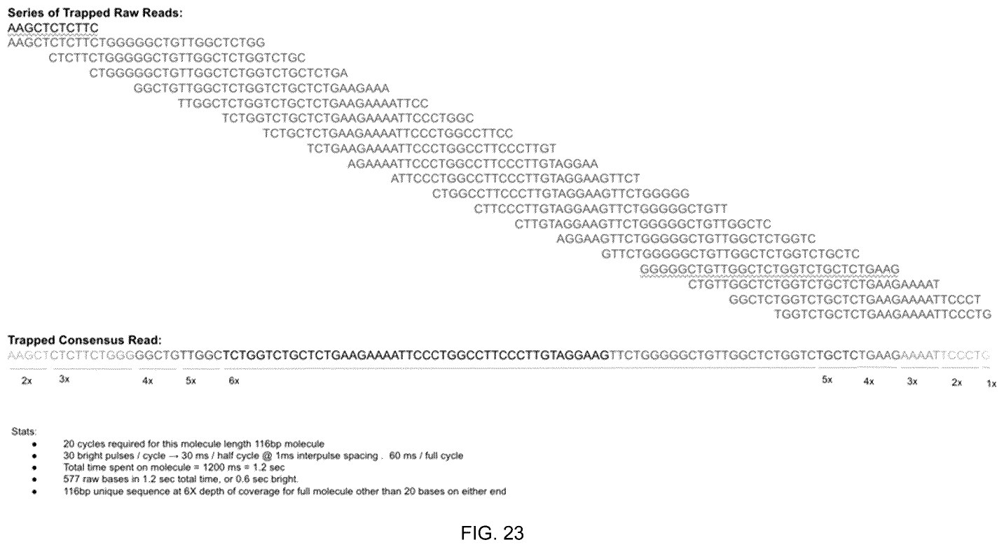

FIG. 23 is an illustration of the assembled trapped raw read series resulting from nanopore sequencing. The read series can be used to generate an intramolecular consensus. The length corresponds to a target nucleic acid molecule that is 116 bp long. The nucleic acid molecule, e.g., a surrogate molecule such as an Xpandomer, was moved with forward cycles of 30 pulses and reverse cycles of 25 pulses through the nanopore. Each pulse moves one nucleotide reading (e.g., corresponding to one or more reporter elements).

In total, 20 cycles were used to cover the entire length of the molecule. The reads for each cycle are shown at the top. Because each cycle includes reads that overlap, individual nucleotides are sequenced several times. The consensus read is shown under “Trapped Consensus Read.” Underneath the trapped consensus read shows the number of times the nucleotide has been sequenced. For example, the initial subsequence of AAGCT is sequenced twice. The middle section starting with TCTGGT is sequenced six times. The beginning of the molecule can be sequenced multiple times if the initial forward and reverse cycles were set to have the same number of pulses before changing to cycles where the bright period has more forward pulses than the dark period has reverse pulses. The end of the molecule can be sequenced multiple times by continuing the forward and reverse pulses until the molecule has fully exited the nanopore.

### VI. Computer System

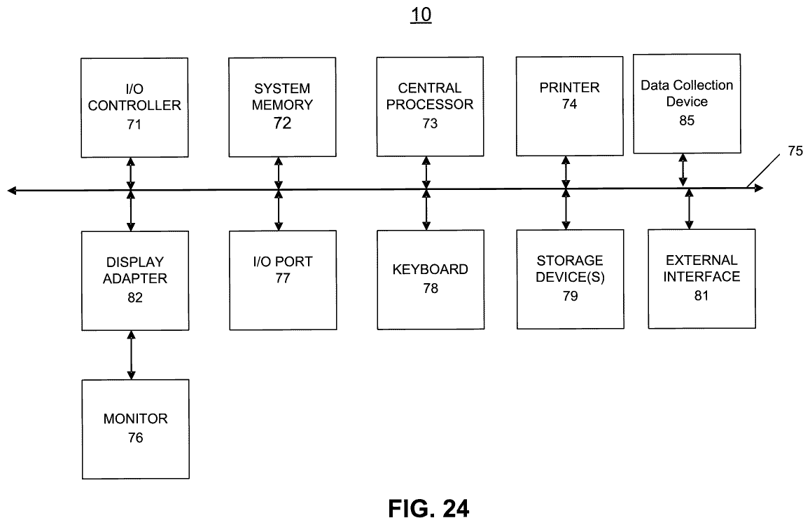

Any of the computer systems mentioned herein may utilize any suitable number of subsystems. Examples of such subsystems are shown in FIG. 24 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 24 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g., Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware (e.g. an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C #, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective steps or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, units, circuits, or other means for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of example embodiments of the invention has been presented for the purposes of illustration and description. It is not intended to be exhaustive or to limit the invention to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover reference to a “first” or a “second” component does not limit the referenced component to a particular location unless expressly stated.

All patents, patent applications, publications, and descriptions mentioned herein are incorporated by reference in their entirety for all purposes. None is admitted to be prior art.

What is claimed is:

1. A method comprising performing, by an inference circuit:
receiving a first stream of raw data from a sensor chip including a plurality of cells, the raw data including a plurality of measurements for each position of a respective nucleic acid molecule of at least 100,000 nucleic acid molecules;
generating a second stream of read data that includes header information, basecall data, and quality scores for the at least 100,000 nucleic acid molecules;
extracting, from the second stream, a first sub-stream of header information that identifies each of the at least 100,000 nucleic acid molecules;
compressing, by a first thread, the first sub-stream of header information to generate compressed header information;
extracting, from the second stream, a second sub-stream of basecall data that provides a basecall at each position of each of the at least 100,000 nucleic acid molecules;
compressing, by a second thread, the second sub-stream of basecall data to generate compressed basecall data;
extracting, from the second stream, a third sub-stream of quality score data that provides a quality score for each basecall at each position of each of the at least 100,000 nucleic acid molecules;
compressing, by a third thread, the third sub-stream of quality score data to generate compressed quality score data; and
outputting the compressed header information, the compressed basecall data, and the compressed quality score data.

2. The method of claim 1, wherein the compressed header information, the compressed basecall data, and the compressed quality score data are combined before outputting.

3. The method of claim 2, wherein combining the compressed header information, the compressed basecall data, and the compressed quality score data are performed using load balancing.

4. The method of claim 1, wherein the basecall data includes a sequence of basecalls for each of the at least 100,000 nucleic acid molecules, and wherein compressing the second sub-stream of basecall data includes:
for each sequence of basecalls corresponding to the respective nucleic acid:
aligning the sequence to a reference sequence to obtain genomic location information;
identifying whether one or more differences exist between the sequence and the reference sequence;
encoding any differences to generate code(s) that specify the difference;
substituting the genomic location information in the reference sequence for at least a portion of the sequence that matches the reference sequence; and
generating the compressed basecall data using the code(s) and the genomic location information.

5. The method of claim 4, wherein the substituted genomic location information specifies a range of genomic locations in the sequence that match the reference sequence.

6. The method of claim 1, wherein the first thread, the second thread, and the third thread execute in series.

7. A method comprising performing, by an inference circuit:
receiving raw data from a sensor chip including a plurality of cells, the raw data including a plurality of measurements for each position of a respective nucleic acid molecule of at least 100,000 nucleic acid molecules, wherein at least a portion of the at least 100,000 nucleic acid molecules include clusters of nucleic acid molecules, wherein the nucleic acid molecules of a cluster correspond to a same template nucleic acid molecule;
for each position of the respective nucleic acid molecule:
determining, using the raw data, a nucleotide at the position, thereby generating a sequence read:
for each sequence read for the at least 100,000 nucleic acid molecules:
identifying a particular cluster corresponding to the sequence read;
incrementing a counter for the particular cluster;
determining that a first counter for a first cluster is greater than a threshold; and
in response to determining that the first counter is greater than the threshold, discarding sequence reads corresponding to the first cluster.

8. The method of claim 7, wherein the sequence reads above the threshold are discarded.

9. The method of claim 7, wherein the sequence read is an intramolecular consensus read.

10. The method of claim 9, wherein the intramolecular consensus read is determined by:
creating a surrogate molecule from the respective nucleic acid molecule, the surrogate molecule including one or more reporter elements corresponding to each nucleotide;
passing the surrogate molecule through a nanopore a plurality of times to obtain a plurality of subreads; and
determining the intramolecular consensus read by comparing the plurality of subreads.

11. The method of claim 7, wherein the sequence read includes one or more barcode sequences corresponding to nucleotides attached to the respective nucleic acid molecule, wherein the particular cluster is assigned to one or more particular barcode sequences, and wherein identifying the particular cluster corresponding to the sequence read includes:
comparing the one or more barcode sequences of the sequence read to the one or more particular barcode sequences to determine a match.

12. The method of claim 11, further comprising:
creating a new cluster for a new sequence read when the one or more barcode sequences of the new sequence read do not match to the one or more particular barcode sequences assigned to existing clusters.

13. The method of claim 7, wherein identifying the particular cluster corresponding to the sequence read includes:
aligning the sequence read to a reference sequence to determine a genomic location; and
comparing the genomic location to an assigned genomic location of the particular cluster.

14. The method of claim 13, wherein the genomic location includes a start genomic location and an end genomic location, and wherein the assigned genomic location of the particular cluster was determined using another sequence read of the particular cluster.

15. The method of claim 7, further comprising:
outputting, form the inference circuit, sequence reads corresponding to the first cluster before the counter is greater than the threshold.

16. The method of claim 7, wherein the particular cluster of nucleic acid molecules is generated by making copies of the same template nucleic acid molecule.

17. The method of claim 16, wherein the copies are generated using PCR.

18. The method of claim 7, further comprising:
generating a consensus sequence read using the sequence reads of the cluster.

19. A system comprising:
a sensor chip including a plurality of sequencing cells, the plurality of sequencing cells including at least 100,000 sequencing cells; and
one or more processors configured to perform:
receiving a first stream of raw data from the sensor chip including the plurality of sequencing cells, the raw data including a plurality of measurements for each position of a respective nucleic acid molecule of at least 100,000 nucleic acid molecules;
generating a second stream of read data that includes header information, basecall data, and quality scores for the at least 100,000 nucleic acid molecules;
extracting, from the second stream, a first sub-stream of header information that identifies each of the at least 100,000 nucleic acid molecules;
compressing, by a first thread, the first sub-stream of header information to generate compressed header information;
extracting, from the second stream, a second sub-stream of basecall data that provides a basecall at each position of each of the at least 100,000 nucleic acid molecules;
compressing, by a second thread, the second sub-stream of basecall data to generate compressed basecall data;
extracting, from the second stream, a third sub-stream of quality score data that provides a quality score for each basecall at each position of each of the at least 100,000 nucleic acid molecules;
compressing, by a third thread, the third sub-stream of quality score data to generate compressed quality score data; and
outputting the compressed header information, the compressed basecall data, and the compressed quality score data.
